(19) United States
(12) Patent Application Publication
(10) Pub. No.: US 2026/0345615 A1
(43) Pub. Date: Nov. 5, 2026

(54) COMPOSITIONS COMPRISING SOTORASIB

(71) Applicant: AMGEN INC., Thousand Oaks, CA (US)

(72) Inventors: Fernando Antonio ALVAREZ-NUNEZ, Newbury Park, CA (US); Sai Prasanth CHAMARTHY, Oak Park, CA (US); Swagat SAHU, Simi Valley, CA (US)

(73) Assignee: AMGEN INC., Thousand Oaks, CA (US)

(21) Appl. No.: 19/487,677

(22) PCT Filed: May 30, 2024
(86) PCT No.: PCT/US2024/031711

§ 371 Date: Nov. 25, 2025

Related U.S. Application Data
(60) Provisional application No. 63/505,374, filed on May 31, 2023.

(51) Int. Cl. A61K 31/519; A61K 9/20; A61K 47/32; A61K 47/38

(52) U.S. Cl. CPC A61K 31/519; A61K 9/2013; A61K 9/2018; A61K 9/2027; A61K 9/2054; A61K 47/32; A61K 47/38

(57) ABSTRACT

Provided herein are amorphous solid dispersions comprising sotorasib in an amorphous form and a matrix polymer. Also provided herein are processes for preparing the disclosed amorphous solid dispersions and pharmaceutical compositions comprising and methods of using the disclosed amorphous solid dispersions.

### CROSS-REFERENCE TO RELATED APPLICATION

This application claims the benefit of priority to U.S. Provisional Patent Application No. 63/505,374, filed May 31, 2023, which is hereby incorporated by reference in its entirety.

### BACKGROUND

KIRSTEN RAT SARCOMA VIRAL ONCOGENE homologue (KRAS) is the most frequently mutated oncogene in human cancers and encodes a guanosine triphosphatase (GTPase) that cycles between active guanosine triphosphate (GTP)—bound and inactive guanosine diphosphate (GDP)—bound states to regulate signal transduction. See, for example, Simanshu D K, Nissley D V, McCormick F. “RAS proteins and their regulators in human disease” in Cell 2017; 170:17-33.

The KRAS G12C mutation occurs in approximately 13% of non-small-cell lung cancers (NSCLCs) and in 1 to 3% of colorectal cancers and other solid cancers. See, for example, Cox A D, Fesik S W, Kimmelman A C, Luo J, Der C J. “Drugging the undruggable RAS: mission possible?” in Nat Rev Drug Discov 2014; 13:828-51; Biernacka A, Tsongalis P D, Peterson J D, et al. “The potential utility of re-mining results of somatic mutation testing: KRAS status in lung adenocarcinoma” in Cancer Genet 2016; 209:195-8; Neumann J, Zeindl-Eberhart E, Kirchner T, Jung A. “Frequency and type of KRAS mutations in routine diagnostic analysis of metastatic colorectal cancer” in Pathol Res Pract 2009; 205:858-62; and Ouerhani S, Elgaaied A B A. “The mutational spectrum of HRAS, KRAS, NRAS and FGFR3 genes in bladder cancer” in Cancer Biomark 2011-2012; 10:259-66.

Sotorasib (also referred to as AMG 510, and marketed commercially as LUMAKRAS® or LUMYKRAS®) is a small molecule that specifically and irreversibly inhibits KRAS^{G12C} and is approved to treat G12C-mutated cancers. Sotorasib has the following chemical structure:

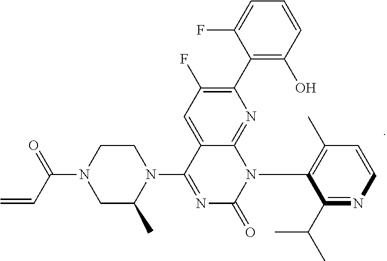

The compound has an atropisomeric chiral center, wherein in the (M)-configuration (shown above) is more active at the target protein than the (P)-configuration.

There remains a need for compositions comprising sotorasib and pharmaceutical compositions comprising sotorasib, which are suitable for patients.

### SUMMARY

The disclosure provides amorphous solid dispersions (ASD), e.g., spray-dried dispersions (SDDs), comprising sotorasib and at least one polymer selected from the group consisting of a cationic acrylate copolymer, a vinylpyrrolidone-acetate copolymer, a cellulose polymer, and a combination thereof.

The disclosure also provides processes for making the disclosed ASDs comprising sotorasib and the at least one polymer.

The disclosure further provides pharmaceutical compositions and methods of using the disclosed ASDs comprising sotorasib and the at least one polymer.

### BRIEF DESCRIPTION OF THE FIGURES

FIG. 1 shows the XRPD of an amorphous solid dispersions (ASD) comprising sotorasib and E PO as described in Example 1.

FIG. 2 shows the XRPD of an amorphous solid dispersions (ASD), comprising sotorasib and PVP-VA as described in Example 1.

FIG. 3 shows the XRPD of an amorphous solid dispersions (ASD) comprising sotorasib and HPMCAS-M as described in Example 1.

FIG. 4 shows the XRPD of spray dried sotorasib as described in Example 1.

FIG. 5 shows the results of the dissolution studies of the amorphous solid dispersions prepared as described in Example 1 as compared to crystalline sotorasib as a reference.

FIG. 6 shows the biorelevant dissolution of sotorasib (amorphous).

FIG. 7 shows the biorelevant dissolution of sotorasib (crystalline)

FIG. 8 shows the biorelevant dissolution of an ASD comprising E PO.

FIG. 9 shows the biorelevant dissolution of an ASD comprising PVP-VA.

FIG. 10 shows the biorelevant dissolution of an ASD comprising HPMCAS-M.

FIG. 11 shows a comparison of the biorelevant dissolution of amorphous and crystalline sotorasib.

FIG. 12 shows a comparison of the biorelevant dissolution of sotorasib (amorphous and crystalline) and ASDs 1-3 from time 0-210 min.

FIG. 13 shows a comparison of the biorelevant dissolution of sotorasib (amorphous and crystalline) and ASDs 1-3 from time 30-210 min.

FIG. 14 shows the dissolution (FaSSIF) of the ASDs 1-3 were compared against amorphous and crystalline sotorasib at T=0 (FIG. 14A), 1 month (FIG. 14B), and 2 months (FIG. 14C).

FIGS. 15A through 15C show the biorelevant dissolution of ASDs 1-3 compared against amorphous and crystalline sotorasib in intestinal fluid and gastric fluid.

FIG. 16 shows the tabletability as relative tensile strength v. the compression pressure of microcrystalline cellulose (MCC Ph 102), lactose, and ASD 3.

FIG. 17 shows the compactibility as relative tensile strength v. solid fraction of microcrystalline cellulose (MCC Ph 102), lactose, and ASD 3.

FIG. 18 shows the dissolution of the 4% ASD 3 blend of Example 4.

FIG. 19 shows the dissolution of the 71% ASD 3 tablet of Example 4.

FIG. 20 shows the dissolution of the 94% ASD 3 blend of Example 4.

### DETAILED DESCRIPTION

The disclosure provides amorphous solid dispersions (ASDs), e.g., spray-dried dispersions (SDDs), comprising sotorasib and at least one polymer, as described herein. Other ASDs include ASDs obtained by hot melt extrusion, freeze drying, rotary evaporation, use of supercritical CO₂ or cosolvent precipitation.

Applicant has discovered that the disclosed ASDs comprising sotorasib provide various advantages over conventional formulations. For example, it has been found that ASDs comprising sotorasib and at least one polymer described herein provide a number of benefits, e.g., unexpectedly good solubility, sustained enhancement in solubility, improved ease of formulation, and/or improved biorelevant dissolution, which may lead to improved bioavailability in vivo. Without wishing to be bound to any particular theory, it is believed that that the ASDs disclosed herein may provide improved bioavailability of sotorasib due to, for example, one or more of the following: 1) improved drug dispersion, thereby preventing or retarding the rate of crystallization in the solid state, 2) improved dissolution in vivo, thereby allowing the drug to be released in the gastrointestinal tract, and/or 3) inhibiting the precipitation or crystallization of aqueous dissolved drug. It is desirable that the ASD comprise sotorasib in a noncrystalline form (i.e., an amorphous form) in the disclosed compositions.

ASDs can be prepared using spray-drying techniques. Spray-drying is a process involving breaking up liquid mixtures into small droplets (atomization) and rapidly removing solvent from the mixture in a container where there is a strong driving force for evaporation of solvent from the droplets. The strong driving force for solvent evaporation is generally provided by maintaining the partial pressure of solvent in the spray-drying apparatus well below the vapor pressure of the solvent at the temperature of the drying droplets. This can be accomplished by (1) maintaining the pressure in the spray-drying apparatus at a partial vacuum (e.g., 0.01-0.50 atm); (2) mixing the liquid droplets with a warm drying gas; or (3) both. For example, a solution of a drug and hydroxypropylmethyl cellulose acetate succinate (HPMCAS) in acetone can be suitably spray-dried by spraying the solution at a temperature of 50 °C (the vapor pressure of acetone at 50 °C is about 0.8 atm) into a chamber held at 0.01-0.2 atm total pressure by connecting the outlet to a vacuum pump. Alternatively, the acetone solution can be sprayed into a chamber where it is mixed with nitrogen or other inert gas at a temperature of 80-180 °C and a pressure of 1.0-1.2 atm. Spray-drying processes and spray-drying equipment are described generally in, for example, Chemical Engineers' Handbook, Sixth Edition (R. H. Perry, D. W. Green, J. O. Maloney, eds.) McGraw-Hill Book Co. 1984, page 20-54 to 20-57. More details on spray-drying processes and equipment are reviewed by Marshall (“Atomization and Spray-Drying,” Chem. Eng. Prog. Monogr. Series, 50 [1954] 2).

The ASD can be used in the preparation of pharmaceutical compositions, e.g., using methods known in the art such as roller compaction, fluid bed agglomeration, or spray coating.

### Sotorasib

The disclosed ASDs comprise sotorasib. Typically, the disclosed ASDs comprise sotorasib in an amorphous form. In some embodiments, sotorasib present in the ASDs is essentially free of crystalline sotorasib. As used herein, the term “essentially free” refers to comprising less than 5% wt % crystalline sotorasib (e.g., 5.0 wt % or less, such as 4.5 wt %, 4.0 wt %, 3.5 wt %, 3.0 wt %, 2.5 wt %, 2.0 wt %, 1.5 wt %, 1.0 wt %, 0.5 wt % or less) of the total amount of sotorasib that is present. In some embodiments, the disclosed amorphous solid dispersions do not include crystalline sotorasib in an amount detectable by X-ray powder diffraction (XRPD), as measured by CuKα=1.54 Å.

Particle Size: The disclosed ASDs have a suitable particle size (e.g., particle size distribution). In some embodiments, in conjunction with other embodiments herein, the ASDs have a D50 of 3 μm to 30 μm (e.g., 3 to 25 μm, 3 to 20 μm, 3 to 15 μm, 3 to 10 μm, 3 to 7 μm, 4 to 6 μm, 4 to 5 μm, 5 to 30 μm, 5 to 25 μm, 5 to 20 μm, 5 to 15 μm, 5 to 10 μm, 10 to 30 μm, 10 to 25 μm, 10 to 20 μm, 10 to 15 μm, 15 to 30 μm, 15 to 25 μm, 15 to 20 μm, 20 to 30 μm, 20 to 25 μm, or 25 to 30 μm). In some embodiments, in conjunction with other embodiments herein, the ASDs have a D50 of 3 μm to 45 μm.

In some embodiments, in conjunction with other embodiments herein, the ASDs have a D10 of 0.7 to 7 μm (e.g., 0.7 to 6 μm, 0.7 to 5 μm, 0.7 to 4 μm, 0.7 to 3 μm, 0.7 to 2 μm, 1 to 2 μm, 3 to 7 μm, 3 to 6 μm, 3 to 5 μm, 3 to 4 μm, 4 to 7 μm, 4 to 6 μm, or 4 to 5 μm). In some embodiments, in conjunction with other embodiments herein, the ASDs have a D10 of 0.7 to 15 μm.

In some embodiments, in conjunction with other embodiments herein, the ASDs have a D90 of 10 to 40 μm (e.g., 10 to 35 μm, 10 to 30 μm, 10 to 25 μm, 10 to 20 μm, 10 to 15 μm, 11-19 μm, 12-18 μm, 13-17 μm, 14-16 μm 15 to 40 μm, 15 to 35 μm, 15 to 30 μm, 15 to 25 μm, 15 to 20 μm, 20 to 40 μm, 25 to 35 μm, 30 to 35 μm, 25 to 40 μm, 25 to 35 μm, 25 to 30 μm, 30 to 40 μm, 30 to 35 μm, or 35 to 40 μm. In some embodiments, in conjunction with other embodiments herein, the ASDs have a D90 of 10 to 97 μm.

In some embodiments, the disclosed ASDs comprise a cationic acrylate copolymer (CACP) and have a a D50 of 3 μm to 10 μm (e.g., 3 to 7 μm, 4 to 6 μm). An illustrative CACP is Eudragit® E100, as described herein. In some embodiments, in conjunction with other embodiments herein, the ASDs comprising CACP have a D10 of 1 μm to 2 μm. In some embodiments, in conjunction with other embodiments herein, the ASDs comprising CACP have a D90 of 10 μm to 20 μm (e.g., 11 to 19 μm, 12 to 18 μm, 13 to 17 μm, 14 to 16 μm, or 15 to 20 μm. Alternatively, or in addition the ASDs comprising CACP can have a D90 of 10 μm, 11 μm, 12 μm, 13 μm, 14 μm, 15 μm, 16 μm, 17 μm, 18 μm, 19 μm, or 20 μm.

In some embodiments, the disclosed ASDs comprise a vinylpyrrolidone-acetate copolymer (VAC). An illustrative VAC is Kollidon® VA 64, as described herein. In these embodiments, the ASDs comprising a VAC typically have a particle size distribution as described herein for the CACP.

In some embodiments, the disclosed ASDs comprise a cellulose polymer and have D50 of 10 μm to 30 μm (e.g., 10 to 25 μm, 15 to 30 μm, or 15 to 20 μm). An illustrative cellulose polymer is hydroxypropylmethyl cellulose acetate succinate, as described herein. In some embodiments, in conjunction with other embodiments herein, the ASDs comprising a cellulose polymer have a D50 of 10 μm, 15 μm, 20 μm, 25 μm, or 30 μm. In some embodiments, the ASDs comprise HPMCAS and have a D50 of 10 μm to 30 μm, as described herein. In some embodiments, in conjunction with other embodiments herein, the ASDs comprising a cellulose polymer (e.g., HPMCAS) have a D10 of 3 μm to 7 μm (e.g., 4 to 6 μm). In some embodiments, in conjunction with other embodiments herein, the ASDs comprising a cellulose polymer (e.g., HPMCAS) have a D90 of 30 μm to 40 μm (e.g., 30 to 35 μm, 35 to 40 μm, or 30, 31, 32, 33, 34, 35, 36, 37, 38, 39, or 40 μm).

Amount: The disclosed ASDs comprise a suitable amount of sotorasib. If the ASD comprises too little sotorasib, the ASD will not have the desired dissolution properties. In addition, if the ASD comprises too much sotorasib, then the ASD will also be too costly or could suffer from other performance issues (e.g., physical instability). In some embodiments, in conjunction with other embodiments herein, the ASDs disclosed herein have a weight ratio of sotorasib to polymer of 1:50 to 10:1 (e.g., 1:10 to 1:1). Further, in various embodiments, the ASD may have a weight ratio of sotorasib to polymer of 1:50, 1:45, 1:40, 1:35, 1:30, 1:25, 1:20, 1:15, 1:10, 1:5, 1:1, 2:1, 3:1, 4:1, 5:1, 6:1, 7:1, 8:1, 9:1, or 10:1). In some embodiments, the weight ratio of sotorasib to the polymer is 1:4. In some embodiments, the weight ratio of sotorasib to the polymer is 1:3.

In some embodiments, in conjunction with other embodiments herein, sotorasib is present in an amount that is 10-80% by weight of the ASD (e.g., 15-75 wt %, 20-70 wt %, 20-65 wt %, 20-60 wt %, 20-55 wt %, 20-50 wt %, 20-45 wt %, 20-40 wt %, 20-35 wt %, or 20-30 wt % of the ASD). In some embodiments, sotorasib is 25 wt % of the ASD.

### Polymers

The ASDs disclosed herein comprise one or more polymers as described herein. Desirably, the polymers of the ASD provide a “matrix” capable of solubilizing an active pharmaceutical ingredient (API) (e.g., sotorasib), while providing a desirable release profile. Suitable polymers include polymers selected from the group consisting of a cationic acrylate copolymer, a vinylpyrrolidone-acetate copolymer, a cellulose polymer, and a combination thereof. In some embodiments, in conjunction with other embodiments herein, the ASDs comprise at least one polymer which comprises a random copolymer.

### Cationic Acrylate Copolymers (CACP)

In some embodiments, the ASDs comprise a cationic acrylate polymer. In various embodiments, the CACP comprises a polymethacrylate polymer. In some embodiments, the CACP comprises a copolymer based on monomers comprising aminoalkyl methacrylate and alkyl methacrylate. Illustrative suitable aminoalkyl methacrylates include for example, dialkylaminoalkyl methacrylates (e.g., 2-dialkylaminoalkyl methacrylates). Illustrative suitable alkyl methacrylates include butyl methacrylate and methyl methacrylate. In some embodiments, the CACP comprises a copolymer based on monomers comprising 2-dimethylaminoethyl methacrylate and an alkyl methacrylate. In some embodiments, the CACP comprises a copolymer based on monomers comprising 2-dimethylaminoethyl methacrylate, butyl methacrylate, and methyl methacrylate.

In some embodiments, in conjunction with other embodiments herein, the cationic acrylate copolymer is based on monomers comprising 2-dimethylaminoethyl methacrylate, butyl methacrylate, and methyl methacrylate with a monomer ratio of 2:1:1. In some embodiments, in conjunction with other embodiments herein, the cationic acrylate copolymer is poly((2-dimethylaminoethyl) methacrylate, butyl methacrylate, methyl methacrylate) (2:1:1).

An illustrative cationic acrylate polymer is Eudragit® E100 available commercially, for example, from Evonik Industries (Essen, Germany). Eudragit® 100 is a cationic copolymer based on dimethylaminoethyl methacrylate, butyl methacrylate, and methyl methacrylate with a ratio of 2:1:1, as follows:

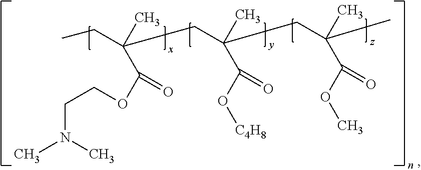

wherein the monomer are randomly distributed along the copolymer chain. Eudragit® E100 is available commercially in several forms (e.g., granules, powder, and liquid) under various tradenames (e.g., Eudragit® E PO and Eudragit® 12,5).

### Vinylpyrrolidone-Acetate Copolymers (VAC)

In some embodiments, the ASDs comprise a vinylpyrrolidone-acetate copolymer. In some embodiments, the VAC comprises a copolymer based on monomers comprising N-vinyl lactam monomers and vinyl acetate monomers. Exemplary N-vinyl lactam monomers include N-vinyl pyrrolidone and N-vinyl caprolactam. Illustrative suitable vinyl acetate monomers include vinyl acetate, vinyl propionate, vinyl butyrate, and vinyl tert-butyrate. In some embodiments, in conjunction with other embodiments herein, the N-vinyl lactam monomers comprise N-vinyl pyrrolidone. In some embodiments, in conjunction with other embodiments herein, the acetate monomers comprise vinyl acetate.

In some embodiments, in conjunction with other embodiments herein, the VAC comprises one or more polymers selected from povidone, copovidone, polyvinyl acetate, and a combination thereof.

In some embodiments, in conjunction with other embodiments herein, the ratio of N-vinyl lactam monomers to acetate monomers is 6:4.

An illustrative VAC is Kollidon® VA 64 available commercially, for example from BASF (Wyandotte, MI) in various forms (e.g., Kollidon® VA 64 or Kollidon® VA 64 Fine).

### Cellulose Polymers

In some embodiments, the ASDs comprise a cellulose polymer. In some embodiments, in conjunction with other embodiments herein, the cellulose polymer is selected from the group consisting of hydroxypropylmethyl cellulose, hydroxypropylmethyl cellulose acetate succinate (HPMCAS), hydroxypropyl methylcellulose phthalate (HPMCP), hydroxypropyl cellulose, and a combination thereof. In some embodiments, the cellulose polymer comprises HPMCAS. In some embodiments, the cellulose polymer is HPMCAS.

As used herein, HPMCAS refers to a family of cellulose derivatives that can have (1) two types of ether substituents, methyl and/or 2-hydroxypropyl and (2) two types of ester substituents, acetyl and/or succinyl. HPMCAS is also known by the chemical name 0-(2-hydroxypropyl)-O-methyl-cellulose acetate succinate. The degree of substitution for each of the four general types just noted can be varied over a wide range to affect the chemical and physical properties of the polymer. This versatility of HPMCAS allows its structure to be optimized to obtain good performance with a particular drug of interest. HPMCAS can be synthesized or purchased commercially. Three examples of commercially available HPMCAS include Shin-Etsu AQOAT®-LF, Shin-Etsu AQOAT®-MF, and Shin-Etsu AQOAT®-HF (Shin-Etsu Chemical Co., Ltd.) wherein L, M and H grades refer to the pH at which the polymers dissolve (L=low pH≥5.5, M=medium pH≥6.0 and H=high pH 6.5). In some embodiments, the HPMCAS is L grade (e.g., HPMCAS-LF or HPMCAS-LG or HPMCAS-LMP). F, G and MP grades refer to differing average particle size (F is fine powder—D50 of not more than 10 μm, MP is medium particle size—D50 is 70-300 μm and G is free-flowing granules—with a median particle size typically around 1000 μm). The L grade has a methoxy %, hydroxpropoxy %, acetyl %, and succinoyl % of 20-24%; 5-9%, 5-9%, and 14-18%, respectively. The M grade has a methoxy %, hydroxpropoxy %, acetyl %, and succinoyl % of 21-25%; 5-9%, 7-11%, and 10-14%, respectively. The H grade has a methoxy %, hydroxpropoxy %, acetyl %, and succinoyl % of 22-26%; 6-10%, 10-14%, and 4-8%, respectively.

The HPMCAS has any suitable molecular weight. In some embodiments, the mean weight-average molecular weight range for HPMCAS is 10,000 to one million daltons (e.g., 10,000 to 400,000 daltons or 55,000 to 115,000 daltons, as determined using polyethylene oxide standards). Note that molecular weight can be presented herein as daltons (Da) or as g/mol, which are used interchangeably throughout. The molecular weight range can also vary based on the degree of substitution (e.g., amount of acetyl and/or succinyl groups present). For example, in various embodiments, in conjunction with other above or below embodiments, the mean weight-average molecular weight of the HPMCAS is approximately 15,000 to 20,000 daltons, for example, 15,000, 16,000, 17,000, 18,000, 19,000, or 20,000 daltons. In some embodiments, mean weight-average molecular weight of the HPMCAS is 17,700, 17,900, 18,800, 18,900, 20,400, or 21,200 daltons. In some embodiments, in conjunction with other above or below embodiments, the number-average molecular weight is approximately 13,000 daltons.

### Process

The disclosure further provides processes for preparing the ASDs disclosed herein. As noted above, ASDs can be prepared via spray-drying, by hot melt extrusion, by freeze drying, by rotary evaporation, use of supercritical CO₂, or by cosolvent precipitation.

In some embodiments, the disclosure provides a process for making a ASD comprising (a) mixing amorphous sotorasib, one or more polymers, and a solvent to form a solution; and (b) spray-drying the solution of step (a), thereby obtaining the spray-dried dispersion. In some embodiments, the disclosure provides a process for making a ASD comprising (a) mixing sotorasib, one or more polymers, and a solvent to form a solution; and (b) spray-drying the solution of step (a), thereby obtaining the spray-dried dispersion. An exemplary process is described in the Examples.

In some embodiments, in conjunction with other embodiments herein, the polymer solution prepared according to the disclosed processes comprise the cationic acrylate copolymers, vinylpyrrolidone-acetate copolymers, cellulose polymers, and a combination thereof, as described herein.

In some embodiments, in conjunction with other embodiments herein, the disclosed processes comprise mixing sotorasib and polymer in a weight ratio from 1:50 to 10:1 (e.g., 1:10 to 1:1), as described herein, that is, 1:50, 1:45, 1:40, 1:35, 1:30, 1:25, 1:20, 1:15, 1:10, 1:5, 1:1, 2:1, 3:1, 4:1, 5:1, 6:1, 7:1, 8:1, 9:1, or 10:1). In some embodiments, the weight ratio of sotorasib to the polymer is 1:4. In some embodiments, the weight ratio of sotorasib to the polymer is 1:3.

### Solvents

The disclosed processes are conducted in a suitable solvent. In some embodiments, the solvent comprises a polar aprotic solvent, for example, acetone, acetonitrile, dichloromethane, dimethylformamide (DMF), dimethylpropleneurea, dimethylsulfoxide (DMSO), ethyl acetate, hexamethylphosphoric triamide, or tetrahydrofuran (THF). In some embodiments, the solvent comprises a polar protic solvent, for example, formic acid, n-butanol, isopropanol, nitromethane, ethanol, methanol, acetic acid, or water. In some embodiments, the solvent comprises a polar aprotic solvent, a polar protic solvent, or a combination thereof. In some embodiments, in conjunction with other above and below embodiments, the solvent is selected from the group consisting of water, methanol, ethanol, isopropanol, acetic acid, acetonitrile, acetone, cyclopentyl methyl ether, ethyl acetate, methyl isobutyl ketone, isopropyl acetate, tetrahydrofuran, methyl tert-butyl ether, N-methylpyrrolidone, dimethylsulfoxide, dimethylformamide, toluene, n-heptane, and a combination thereof. In some embodiments, the solvent is selected from the group consisting of water, methanol, ethanol, isopropanol, acetic acid, acetone, acetonitrile, dichloromethane, dimethyformamide, N-methylpyrrolidone, dimethylsulfoxide, and a combination thereof. In some embodiments, the solvent is acetone, dichloromethane, or a mixture thereof.

The solvent is present in a suitable amount. If the amount of solvent present is too little, then the reactants may not mix in a suitable manner to facilitate the reaction or processing of the reaction mixture. In contrast, if the amount of solvent is too high, then the concentration of the reactants may be too dilute for suitable reaction or the processing of the reaction may be unnecessarily cumbersome or have high energy requirements to remove solvent.

In various embodiments, the disclosure provides ASDs prepared according to the disclosed processes.

### Pharmaceutical Compositions

In some embodiments, the disclosed ASDs are administered in the form of a pharmaceutical composition. In these embodiments, a pharmaceutical composition comprises a ASD and a pharmaceutically acceptable excipient. Suitable pharmaceutically acceptable excipients include, for example, vehicles, adjuvants, and diluents, which are well known to those who are skilled in the art and which are readily available. Typically, the pharmaceutically acceptable excipient is one that is chemically inert to the active compounds and one that has no detrimental side effects or toxicity under the conditions of use. In some embodiments, in conjunction with other embodiments herein, the pharmaceutical acceptable excipient is a diluent, a disintegrant, a lubricant, or a combination thereof.

In some embodiments, the disclosed pharmaceutical compositions comprise a disintegrant. Exemplary disintegrants include, but are not limited to, cross-linked sodium carboxy methyl cellulose (croscarmellose sodium), cross-linked polyvinylpyrrolidone (crospovidone), sodium starch glycolate, pregelatinized starch, calcium carboxymethyl cellulose, low substituted hydroxypropyl cellulose, and magnesium aluminum silicate, and combinations thereof. In some embodiments, the disintegrant comprises croscarmellose sodium or sodium starch glycolate or both. The disintegrant can comprise croscarmellose sodium, crospovidone, sodium starch glycolate, pregelatinized starch, calcium carboxymethyl cellulose, low substituted hydroxypropyl cellulose, magnesium aluminum silicate, or a combination thereof. In some embodiments, the disintegrant comprises croscarmellose sodium, sodium starch glycolate, or a combination thereof. In some embodiments, the disintegrant is croscarmellose sodium.

In some embodiments, the disclosed pharmaceutical compositions comprise a diluent. In some embodiments, the diluent comprises lactose, dibasic calcium phosphate (DCP), mannitol, sorbitol, xylitol, calcium carbonate, magnesium carbonate, tribasic calcium phosphate, trehalose, microcrystalline cellulose, starch, or a combination thereof. In some embodiments, the diluent comprises lactose, dibasic calcium phosphate (DCP), mannitol, microcrystalline cellulose, starch, or a combination thereof. In some embodiments, the diluent comprises lactose, microcrystalline cellulose, or a combination thereof. In some embodiments, the diluent comprises lactose, starch, and a combination thereof. In some embodiments, the starch is pregelatinized starch or corn starch. In some embodiments, the diluent comprises lactose, dibasic calcium phosphate (DCP), mannitol, or a combination thereof. In some embodiments, the lactose is lactose monohydrate.

In some embodiments, the disclosed pharmaceutical compositions comprise a lubricant. Exemplary lubricants include, but are not limited to, magnesium stearate, calcium stearate, oleic acid, caprylic acid, stearic acid, magnesium isovalerate, calcium laurate, magnesium palmitate, behenic acid, glyceryl behenate, glyceryl stearate, sodium stearyl fumarate, potassium stearyl fumarate, zinc stearate, sodium oleate, sodium stearate, sodium benzoate, sodium acetate, sodium chloride, talc, polyethylene glycol, and hydrogenated vegetable oil. In some embodiments, the lubricant is magnesium stearate. In some embodiments, in conjunction with other embodiments herein, the lubricant comprises magnesium stearate, calcium stearate, oleic acid, caprylic acid, stearic acid, magnesium isovalerate, calcium laurate, magnesium palmitate, behenic acid, glyceryl behenate, glyceryl stearate, sodium stearyl fumarate, potassium stearyl fumarate, zinc stearate, sodium oleate, sodium stearate, sodium benzoate, sodium acetate, sodium chloride, talc, polyethylene glycol, hydrogenated vegetable oil, or a combination thereof.

The disclosed pharmaceutical compositions can be administered in various forms, including for example, tablets, capsules, granules, powders, solutions, suspensions, and emulsions. The dosage forms of the compositions can be tailored to the desired mode of administration of the compositions. For oral administration, the compositions can take the form of, e.g., a tablet or capsule (including softgel capsule), or can be, e.g., an aqueous or nonaqueous solution, suspension or syrup. Tablets and capsules for oral administration can include one or more excipients, diluents and carriers, as described herein. If desired, flavoring, coloring and/or sweetening agents can be added to the solid and liquid formulations. Other optional ingredients for oral formulations include without limitation preservatives, suspending agents, and thickening agents. Oral formulations can also have an enteric coating to protect the ASD from the acidic environment of the stomach.

### Methods of Use

In some embodiments, the disclosure provides methods of treating cancer using a pharmaceutical composition as disclosed herein. In some embodiments, one or more cells of the cancer express a KRAS G12C mutant protein, i.e., the cancer is mediated by a KRAS G12C mutation. Exemplary cancers that can be treated as described herein include, for example, non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, brain cancer, or melanoma. In some embodiments, the cancer is non-small cell lung cancer, colorectal cancer, pancreatic cancer, appendiceal cancer, endometrial cancer, cancer of unknown primary, ampullary cancer, gastric cancer, small bowel cancer, sinonasal cancer, bile duct cancer, or melanoma. In some embodiments, the cancer is non-small cell lung cancer. In some embodiments, the cancer is colorectal cancer. In some embodiments, the cancer is pancreatic cancer. In some embodiments, the cancer is brain cancer. In some embodiments, the cancer is one or more brain metastasis originating from a primary cancer, e.g., non-small cell lung cancer. In some embodiments, the cancer is KRAS G12C-mutated locally advanced or metastatic non-small cell lung cancer, as determined by a suitable test, such as an US Food and Drug Administration (FDA)-approved test, who have received at least one prior systemic therapy. In some embodiments, the patient is an adult.

In some embodiments, the disclosure provides methods of treating cancer in a patient comprising administering to the patient a therapeutically effective amount of sotorasib provided in the form of the ASD disclosed herein or as a pharmaceutical composition as disclosed herein.

### EMBODIMENTS

1. An amorphous solid dispersion (ASD) comprising sotorasib and at least one polymer selected from the group consisting of a cationic acrylate copolymer, a vinylpyrrolidone-acetate copolymer, a cellulose polymer, and a combination thereof.

2. The ASD of embodiment 1, wherein sotorasib is essentially free of crystalline sotorasib.

3. The ASD of embodiment 1 or 2, which is devoid of crystalline sotorasib in an amount detectable by XRPD.

4. The ASD of any one of embodiments 1-3, wherein the at least one polymer comprises a cationic acrylate copolymer.

5. The ASD of embodiment 4, wherein the cationic acrylate copolymer comprises a polymethacrylate polymer.

6. The ASD of any one of embodiments 1-5, wherein the cationic acrylate copolymer comprises a copolymer based on monomers comprising aminoalkyl methacrylate and alkyl methacrylate.

7. The ASD of any one of embodiments 1-6, wherein the cationic acrylate copolymer comprises a copolymer based on monomers comprising 2-dimethylaminoethyl methacrylate and an alkyl methacrylate.

8. The ASD of any one of embodiments 1-7, wherein the cationic acrylate copolymer comprises a copolymer based on monomers comprising 2-dimethylaminoethyl methacrylate, butyl methacrylate, and methyl methacrylate.

9. The ASD of embodiment 8, wherein the cationic acrylate copolymer is based on monomers comprising 2-dimethylaminoethyl methacrylate, butyl methacrylate, and methyl methacrylate with a monomer ratio of 2:1:1.

10. The ASD of any one of embodiments 1-9, wherein the cationic acrylate copolymer is poly((2-dimethylaminoethyl) methacrylate, butyl methacrylate, methyl methacrylate) (2:1:1).

11. The ASD of any one of embodiments 1-10, wherein the vinylpyrrolidone-acetate copolymer comprises a copolymer based on monomers comprising N-vinyl lactam monomers and vinyl acetate monomers.

12. The ASD of embodiment 11, wherein the N-vinyl lactam monomers comprise N-vinyl pyrrolidone.

13. The ASD of embodiment 11 or 12, wherein the acetate monomers comprise vinyl acetate.

14. The ASD of any one of embodiments 11-13, wherein the polymer comprises one or more polymers selected from povidone, copovidone, polyvinyl acetate, and a combination thereof.

15. The ASD of any one of embodiments 11-14, wherein the ratio of N-vinyl lactam monomers to acetate monomers is 6:4.

16. The ASD of any one of embodiments 1-22, wherein the at least one polymer comprises a random copolymer.

17. The ASD of any one of embodiments 1-23, wherein the at least one polymer comprises one or more cellulose polymer selected from the group consisting of hydroxypropylmethyl cellulose, hydroxypropylmethyl cellulose acetate succinate, hydroxypropyl methylcellulose phthalate (HPMCP), hydroxypropyl cellulose, and a combination thereof.

18. The ASD of embodiment 24, wherein the cellulose polymer comprises hydroxypropylmethyl cellulose acetate succinate.

19. The ASD of any one of embodiments 1-16, wherein the ASD has a D50 of 3 μm to 10 μm.

20. The ASD of embodiment 19, wherein the D50 is 3 μm to 7 μm.

21. The ASD of embodiment 20, wherein the D50 is 4 μm to 6 μm.

22. The ASD of any one of embodiments 19-21, wherein the ASD has a D10 of 1 μm to 2 μm.

23. The ASD of any one of embodiments 19-22, wherein the ASD has a D90 of 10 μm to 20 μm.

24. The ASD of embodiment 17 or 18, wherein the ASD has a D50 of 10 μm to 30 μm.

25. The ASD of embodiment 24, wherein the ASD has a D50 of 15 μm to 20 μm.

26. The ASD of embodiment 24 or 25, wherein the ASD has a D10 of 3 μm to 7 μm.

27. The ASD of embodiment 26, wherein the ASD has a D90 of 30 μm to 40 μm. Further provided herein as alternative Embodiment 27 is the ASD of embodiment 26, wherein the ASD has a D90 of 30 μm to 50 μm.

28. The ASD of any one of embodiments 1-27, having a weight ratio of sotorasib to the polymer of 1:50 to 10:1.

29. The ASD of embodiment 28, wherein the weight ratio of sotorasib to the polymer is 1:10 to 1:1.

30. The ASD of embodiment 29, wherein the weight ratio of sotorasib to the polymer is 1:4. Further provided herein as alternative embodiment 30 is the ASD of embodiment 29, wherein the weight ratio of sotorasib to the polymer is 1:3.

31. The ASD of any one of embodiments 1-30, wherein sotorasib is 10-80% by weight of the ASD.

32. The ASD of embodiment 31, wherein sotorasib is 25 wt % of the ASD.

33. A process for making an amorphous drug dispersion (ASD) comprising:
(a) mixing amorphous sotorasib, one or more polymers, and a solvent to form a solution; and
(b) spray-drying the solution of step (a), thereby obtaining the ASD.
Further provided herein as alternative embodiment 33 is a process for making an amorphous drug dispersion (ASD) comprising:
(a) mixing sotorasib, one or more polymers, and a solvent to form a solution; and
(b) spray-drying the solution of step (a), thereby obtaining the ASD.

34. The process of embodiment 33, wherein the solvent comprises acetone, dichloromethane, or a mixture thereof.

35. The process of embodiment 33 or 34, wherein the one or more polymers is selected from the group consisting of a cationic acrylate copolymer, a vinylpyrrolidone-acetate copolymer, a cellulose polymer, and a combination thereof.

36. The process of any one of embodiments 33-35, wherein sotorasib and the polymer is present in a weight ratio from 1:50 to 10:1.

37. The process of embodiment 36, wherein the weight ratio of sotorasib to polymer is from 1:10 to 1:1.

38. The process of embodiment 37, wherein the weight ratio of sotorasib to the polymer is 1:4. Further provided herein as alternative embodiment 38 is the process of embodiment 37, wherein the weight ratio of sotorasib to the polymer is 1:4.

39. The process of any one of embodiments 33-38, wherein the ASD comprises sotorasib in an amount from 10-80% by weight of the ASD.

40. The process of embodiment 39, wherein the ASD comprises sotorasib in an amount from 15-50% by weight of the ASD.

41. The process of embodiment 40, wherein the ASD comprises sotorasib in an amount of 25% by weight ASD.

42. An ASD prepared by the process according to any one of embodiments 33-41.

43. The ASD of embodiment 42, which is essentially free of crystalline sotorasib.

44. The ASD of embodiment 42, which is devoid of crystalline sotorasib in an amount detectable by XRPD.

45. A pharmaceutical composition comprising the ASD of any one of embodiments 1-32 and 42-44 and a pharmaceutically acceptable excipient.

46. The pharmaceutical composition of embodiment 45, wherein the pharmaceutically acceptable excipient is a diluent, a disintegrant, a lubricant, or a combination thereof.

47. The pharmaceutical composition of embodiment 46, wherein the diluent comprises lactose, dibasic calcium phosphate (DCP), mannitol, sorbitol, xylitol, calcium carbonate, magnesium carbonate, tribasic calcium phosphate, trehalose, microcrystalline cellulose, starch, or a combination thereof.

48. The pharmaceutical composition of embodiment 47, wherein the diluent comprises lactose, dibasic calcium phosphate (DCP), mannitol, microcrystalline cellulose, starch, or a combination thereof.

49. The pharmaceutical composition of embodiment 48, wherein the diluent comprises lactose, microcrystalline cellulose, or a combination thereof.

50. The pharmaceutical composition of embodiment 48, wherein the diluent comprises lactose, starch, and a combination thereof.

51. The pharmaceutical composition of embodiment 48, wherein starch is pregelatinized starch or corn starch.

52. The pharmaceutical composition of embodiment 48, wherein the diluent comprises lactose, dibasic calcium phosphate (DCP), mannitol, or a combination thereof.

53. The pharmaceutical composition of any one of embodiments 47-52, wherein lactose is lactose monohydrate.

54. The pharmaceutical composition of any one of embodiments 46-53, wherein the disintegrant comprises cross-linked sodium carboxy methyl cellulose (croscarmellose sodium), cross-linked polyvinylpyrrolidone (crospovidone), sodium starch glycolate, pregelatinized starch, calcium carboxymethyl cellulose, low substituted hydroxypropyl cellulose, magnesium aluminum silicate, or a combination thereof.

55. The pharmaceutical composition of embodiment 54, wherein the disintegrant comprises croscarmellose sodium, sodium starch glycolate, or a combination thereof.

56. The pharmaceutical composition of embodiment 54, wherein the disintegrant is croscarmellose sodium.

57. The pharmaceutical composition of any one of embodiments 46-56, wherein the lubricant comprises magnesium stearate, calcium stearate, oleic acid, caprylic acid, stearic acid, magnesium isovalerate, calcium laurate, magnesium palmitate, behenic acid, glyceryl behenate, glyceryl stearate, sodium stearyl fumarate, potassium stearyl fumarate, zinc stearate, sodium oleate, sodium stearate, sodium benzoate, sodium acetate, sodium chloride, talc, polyethylene glycol, hydrogenated vegetable oil, or a combination thereof.

58. The pharmaceutical composition of embodiment 57, wherein the lubricant is magnesium stearate.

59. A method of treating cancer in a patient, the method comprising administering to the patient a therapeutically effective amount of sotorasib provided as the ASD of any one of embodiments 1-32 and 42-44, or the pharmaceutical composition of any one of embodiments 45-58.

60. The method of embodiment 59, wherein one or more cells of the cancer express a KRAS G12C mutant protein.

61. The method of embodiment 59 or 60, wherein the cancer is non-small cell lung cancer, colorectal cancer, pancreatic cancer, appendiceal cancer, endometrial cancer, cancer of unknown primary, ampullary cancer, gastric cancer, small bowel cancer, sinonasal cancer, bile duct cancer, or melanoma.

62. A pharmaceutical composition comprising the ASD of any one of embodiments 1-32 and 42-44 and a pharmaceutically acceptable excipient.

63. The pharmaceutical composition of embodiment 62, wherein the pharmaceutical composition comprises a disintegrant.

64. The pharmaceutical composition of embodiment 63, wherein the disintegrant comprises cross-linked sodium carboxy methyl cellulose (croscarmellose sodium), cross-linked polyvinylpyrrolidone (crospovidone), sodium starch glycolate, pregelatinized starch, calcium carboxymethyl cellulose, low substituted hydroxypropyl cellulose, magnesium aluminum silicate, or a combination thereof.

65. The pharmaceutical composition of embodiment 64, wherein the disintegrant comprises croscarmellose sodium, sodium starch glycolate, or a combination thereof.

66. The pharmaceutical composition of embodiment 65, wherein the disintegrant is croscarmellose sodium.

67. The pharmaceutical composition of any one of embodiments 63-66, wherein the pharmaceutical composition comprises 2-10% (w/w) of the disintegrant.

68. The pharmaceutical composition of any one of embodiments 63-66, wherein the pharmaceutical composition comprises 6% (w/w) of the disintegrant and 94% (w/w) of the ASD.

69. The pharmaceutical composition of embodiment 67, wherein the pharmaceutical composition comprises 3% (w/w), 3.5% (w/w), or 6% (w/w) of the disintegrant.

70. The pharmaceutical composition of any one of embodiments 62-67 and 69, wherein the pharmaceutical composition comprises a diluent.

71. The pharmaceutical composition of embodiment 70, wherein the diluent comprises lactose, dibasic calcium phosphate (DCP), mannitol, sorbitol, xylitol, calcium carbonate, magnesium carbonate, tribasic calcium phosphate, trehalose, microcrystalline cellulose, starch, or a combination thereof.

72. The pharmaceutical composition of embodiment 71, wherein the diluent comprises lactose, dibasic calcium phosphate (DCP), mannitol, microcrystalline cellulose, starch, or a combination thereof.

73. The pharmaceutical composition of embodiment 72, wherein the diluent comprises lactose, microcrystalline cellulose, or a combination thereof.

74. The pharmaceutical composition of any one of embodiments 70-73, wherein the pharmaceutical composition comprises 1-95% (w/w) of the diluent.

75. The pharmaceutical composition of embodiment 74, wherein the pharmaceutical composition comprises 92% (w/w) or 25% (w/w) of the diluent.

76. The pharmaceutical composition of any one of embodiments 70-75, wherein the pharmaceutical composition comprises 25% (w/w) of lactose.

77. The pharmaceutical composition of any one of embodiments 70-75, wherein the pharmaceutical composition comprises 23% (w/w) of lactose and 69% (w/w) microcrystalline cellulose.

78. The pharmaceutical composition of any one of embodiments 62-67 and 69-77, wherein the pharmaceutical composition comprises a lubricant.

79. The pharmaceutical composition of embodiment 78, wherein the lubricant comprises magnesium stearate, calcium stearate, oleic acid, caprylic acid, stearic acid, magnesium isovalerate, calcium laurate, magnesium palmitate, behenic acid, glyceryl behenate, glyceryl stearate, sodium stearyl fumarate, potassium stearyl fumarate, zinc stearate, sodium oleate, sodium stearate, sodium benzoate, sodium acetate, sodium chloride, talc, polyethylene glycol, hydrogenated vegetable oil, or a combination thereof.

80. The pharmaceutical composition of embodiment 78, wherein the lubricant is magnesium stearate.

81. The pharmaceutical composition of any one of embodiments 78-80, wherein the pharmaceutical composition comprises 0.5-5% (w/w) of the lubricant.

82. The pharmaceutical composition of embodiment 81, wherein the pharmaceutical composition comprises 0.6% (w/w) or 1% (w/w) of the lubricant.

83. The pharmaceutical composition of embodiment 62, wherein the pharmaceutical composition comprises 94% (w/w) of ASD 3 and 6% (w/w) of croscarmellose sodium.

84. The pharmaceutical composition of embodiment 62, wherein the pharmaceutical composition comprises 70.7% (w/w) of ASD 3, 25.2% (w/w) of lactose, 3.5% (w/w) of croscarmellose sodium, and 0.6% (w/w) of magnesium stearate.

85. The pharmaceutical composition of embodiment 62, wherein the pharmaceutical composition comprises 4% (w/w) of ASD 3, 69% (w/w) of microcrystalline cellulose, 23% (w/w) of lactose, 3% (w/w) of croscarmellose sodium, and 1% (w/w) of magnesium stearate.

86. A method of treating cancer in a patient, the method comprising administering to the patient a therapeutically effective amount of sotorasib provided as the ASD of any one of embodiments 1-32 and 42-44, or the pharmaceutical composition of any one of embodiments 62-85.

87. The method of embodiment 86, wherein one or more cells of the cancer express a KRAS G12C mutant protein.

88. The method of embodiment 86 or 87, wherein the cancer is non-small cell lung cancer, colorectal cancer, pancreatic cancer, appendiceal cancer, endometrial cancer, cancer of unknown primary, ampullary cancer, gastric cancer, small bowel cancer, sinonasal cancer, bile duct cancer, or melanoma.

89. The method of embodiment 88, wherein the cancer is non-small cell lung cancer.

90. The method of embodiment 88, wherein the cancer is colorectal cancer.

91. The method of embodiment 88, wherein the cancer is pancreatic cancer.

### EXAMPLES

The following examples further illustrate the disclosed processes, but of course, should not be construed as in any way limiting their scope.

The following abbreviations are used herein: SL/M refers to standard liter per minute; FaSSIF refers to fasted state simulated intestinal fluid; FaSSGF refers to fasted state gastric fluid; HPLC refers to high performance liquid chromatography; SDD refers to spray-dried dispersion; DAD refers to diode array detector; HPMCAS refers to hydroxypropyl methyl cellulose acetyl succinate; XRPD refers to X-ray powder diffraction; DSC refers to differential scanning calorimetry; Tg refers to glass transition temperature, TGA refers to thermal gravimetric analysis, and PSD refers to particle size distribution.

### Example 1—Preparation of Solid Dispersions Comprising Sotorasib

Solid dispersions comprising sotorasib at a 25% drug load were prepared using Eudragit™ E PO, polyvinyl pyrrolidone/vinylacetate (PVP-VA), and HPMCAS-M. These samples were characterized as amorphous by XRPD and their glass transition temperatures were measured by differential scanning. The dispersions were prepared using the following spray-drying procedure.

General Procedure for Spray-Drying: The following is an illustrative procedure for preparing spray-drying samples using a Buchi 290 (Buchi Corporation, New Castle, Delaware).

Assembly: Prepare a solution of (a) 1.25 g of sotorasib and (b) 3.75 g of a polymer together in 100 mL of a solvent (e.g., dichloromethane or 1:1 dichloromethane:acetone). Table 1 below shows three ASDs prepared using different polymers with sotorasib via this procedure.
Connect drying chamber with o-ring, outlet facing the cyclone, and secure using the black handle from the left. Connect the collection vessel.
Connect chamber to cyclone through a metal connector with a temp probe port. Connect and secure the cyclone top outlet with a white plastic connector. Connect to chiller.
Insert nozzle on top of the drying chamber, connect N₂ flow by screwing (red tag), connect cooling tubes (left side, WK 230 Lauda Brinkmann)
Secure syringe with solution/solvent

### Operation:

Turn on chiller, air (left side, right blue line), and N₂ (right side). Oxygen level should be less than 1% on the left red monitor once the system is properly assembled. Red light will turn green.
Turn on spray dryer and set N₂ to approximately 0.5% (right side, back, right knob)

### Parameters for Sotorasib Spray Drying

Set N₂ to 8-10 SLIM using the front knob.
Inlet temp: 70-90 °C (On). Follow outlet temp.
Outlet temp: 60-70 °C
Acetone/dichloromethane solution (about 5 mL): manually injected
Spray solution (about 100 mL): 1-1.5 mL/min
Aspirator: 100%
Needle size: 1-2 mm
Nozzle size: 1.5-2.8 mm

### Secondary Drying

Turn off nitrogen and then aspirator before collecting collection vessel.
Loosely cover vessel with aluminum foil and place in vac oven in 1044 at 60 °C and 150 mBar overnight.

TABLE 1
ASDs of sotorasib and polymer.

| ASD | Polymer | D10 (μm) | D50 (μm) | D90 (μm) |
|---|---|---|---|---|
| 1 | E PO | <2.0 | <6.0 | <13 |
| 2 | PVP-VA | <1.0 | <4.0 | <12 |
| 3 | HPMCAS-M | <4.0 | <17 | <37 |
| control | sotorasib amorphous | <1.0 | <4.0 | <16 |

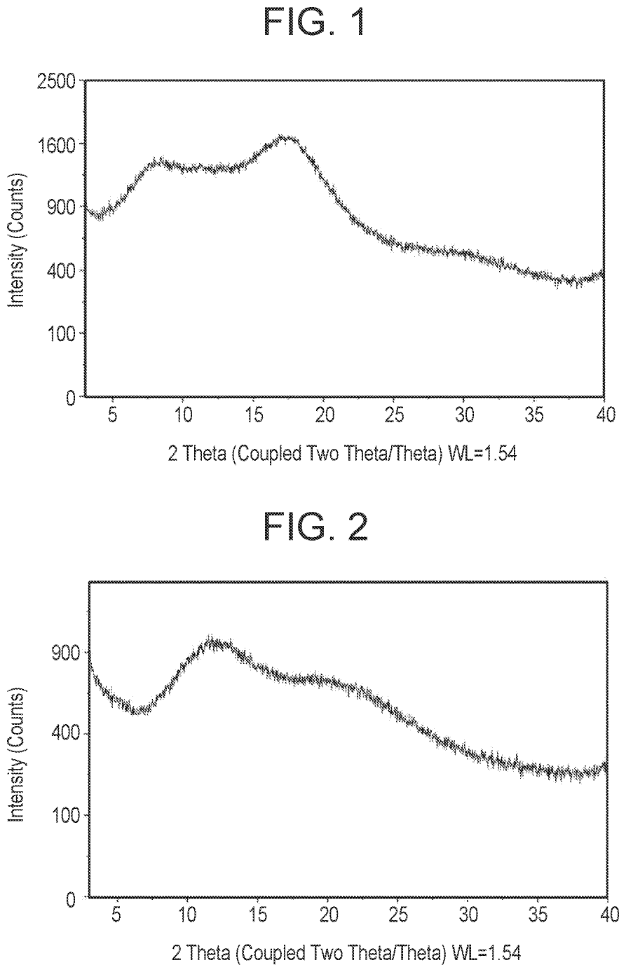

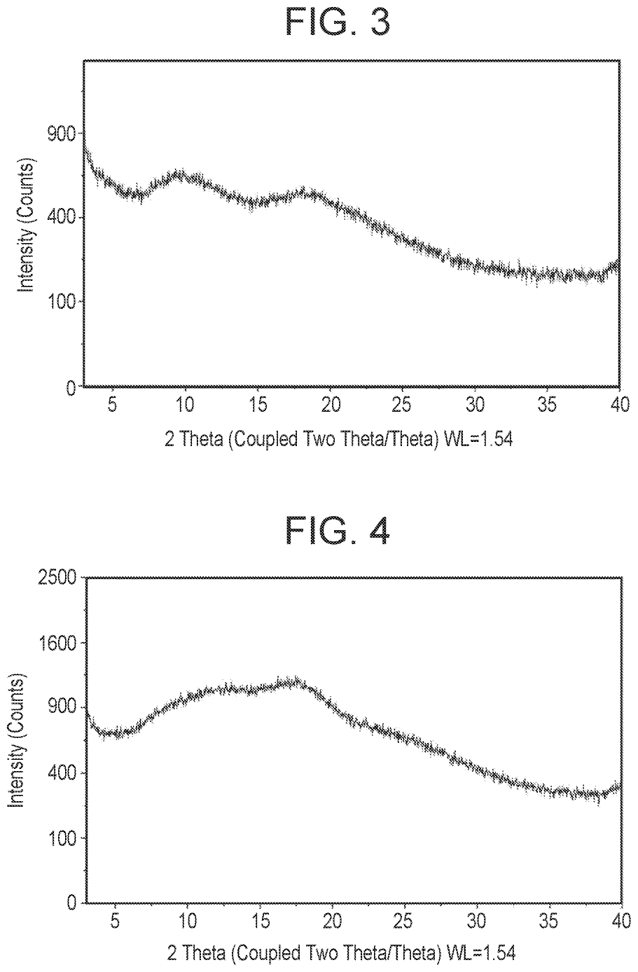

The results of the XRPD analyses of the ASDs in Table 1 are shown in FIGS. 1-4 (XRPD). The results of the XRPD and DSC demonstrate that the components of each of the dispersions were amorphous, that is, no crystalline material could be detected by XRPD. Moreover, the DSC analysis demonstrated that the glass transition temperature (Tg) for each of the compositions was greater than 100 °C. For example, the Tg measured for the ASD 1 were 57 °C and 123 °C, ASD's 2 and 3 were 120 °C.

Differential Scanning Calorimetry (DSC) Analysis: DSC data is collected using a TA Instruments Q1000 DSC. Approximately, 2-6 mg of the sample was placed in a Tzero aluminum pan and sealed with Tzero lid and was equilibrated at 4 °C for 5 min and about the temperature was modulated ±2 °C every 60 seconds while scanning from about 4 °C to 220 °C at a rate of about 4 °C/min under a nitrogen purge of about 50 mL/min.

X-Ray Powder Diffraction (XRPD) Analysis: XRPD data was obtained using a Bruker D8 Advance diffractometer. Samples were scanned at room temperature (25 °C) in continuous mode from 3-40 degrees (2θ) with step size of 0.02 degrees at 40 kV and 40 mA with CuKα radiation (1.54 Å). The incident beam path was equipped with a primary soller slit 2.5 degree and a divergence slit 0.6 mm, in a fixed slit mode. Samples were prepared on a low background sample holder and placed on a spinning stage with a rotation time of 10 rev/min.

Particle Size Distribution (PSD): Analysis of each of the samples was obtained using a Sympatec Helos laser diffraction analyzer. The results of the particle size analysis are shown in Table 1 (D10, D50, and D90 values).

All of the ASDs exhibited enhanced dissolution and solubility relative to crystalline sotorasib in FASSIF as shown by the following micro-dissolution experiment where the dissolved material was quantified by HPLC.

Dissolution: The dissolution properties of ASDs 1-3 and the control spray-dried sotorasib were assessed as described herein. 2.5 mg of amorphous sotorasib (control) and 10 mg of ASDs1-3 as listed in Table 1 were added to separate 10 mL vials of FASSIF (pH 6.50) and samples were collected at 5, 10, 15, 20, 30, 60, and 120 min timepoints. Each sample was diluted by 5-fold in acetonitrile and run on HPLC using a C18 column as the stationary phase under the conditions shown in Table 2. The mobile phase was water and acetonitrile with 0.1% trifluoroacetic acid. The method was a gradient of 5% acetonitrile 95% water to 98% acetonitrile 2% water over 15 minutes using a wavelength 254 nm for detection and quantification.

TABLE 2
HPLC method for dissolution studies.

| HPLC System: | | Waters | |
|---|---|---|---|
| Data Capture System: | | Empower | |
| Column: | | Waters YMC Pro C18, 100 × 3.0 mm, 3 μm | |
| Mobile Phase A: | | Water (0.1% TFA) | |
| Mobile Phase B: | | Acetonitrile (0.1% TFA) | |
| Time | Flow | Mobile Phase A | Mobile Phase B |
| (min) | (mL/min) | (%) | (%) |
| 0.0 | 0.5 | 95 | 5 |
| 0.5 | | 95 | 5 |
| 7.5 | | 2 | 98 |
| 10.5 | | 2 | 98 |
| 11.0 | | 95 | 5 |
| 15.0 | | 95 | 5 |

| | |
|---|---|
| Channel Monitoring | Pressure |
| Draw Position: | 3 mm |
| Sample Tray Temperature: | Set at 5 °C |
| Column Temperature: | 30 °C |
| Injection Volume: | 5 μL |
| Flow rate: | 0.5 mL/min |
| Bandwidth: | 4 nm |
| DAD: | On, Step = 4, 200-600 nm |
| UV Detection Wavelength: | 254 nm |
| Run Time: | 15 min |

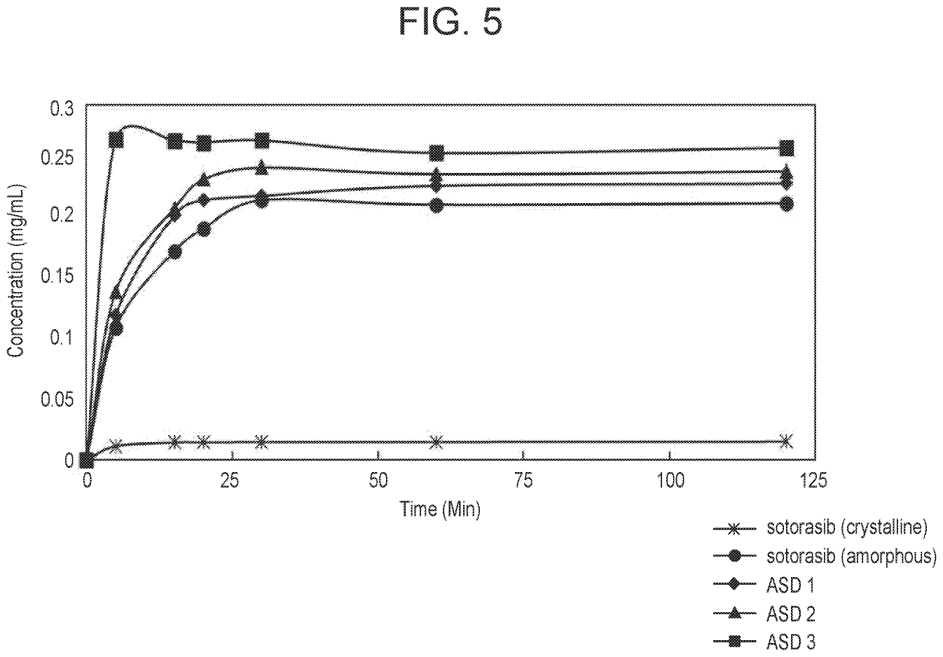

The dissolution of the ASDs 1-3 were compared against amorphous and crystalline sotorasib (AMG 510) (Form I, see WO2020/236947). The results of the dissolution study are shown in FIG. 5 and demonstrate that each of the ASDs prepared demonstrated enhanced dissolution and solubility compared to the crystalline sotorasib, wherein the ASD comprising sotorasib and HPMCAS-M exhibited the highest dissolution.

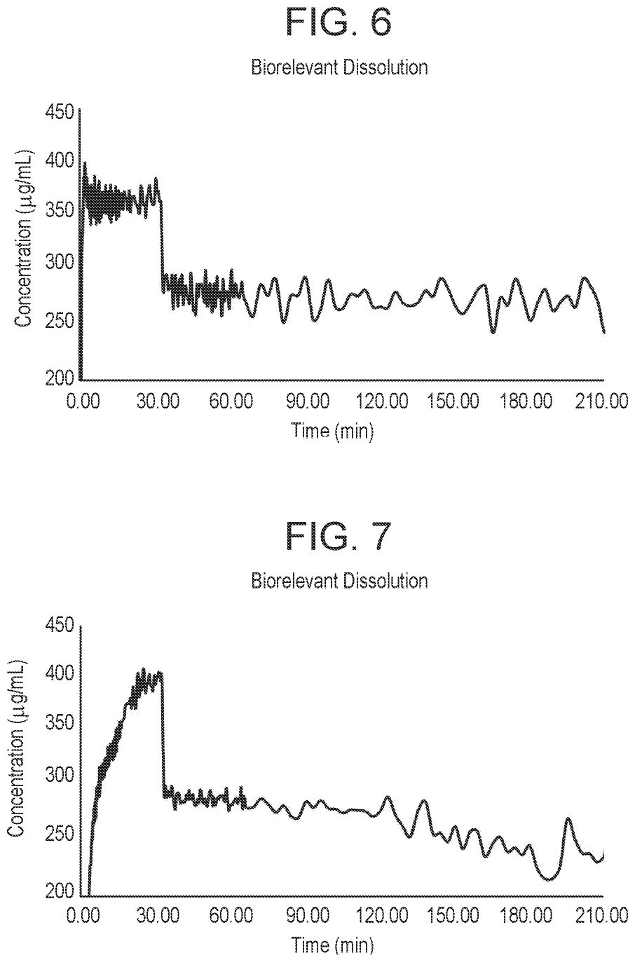

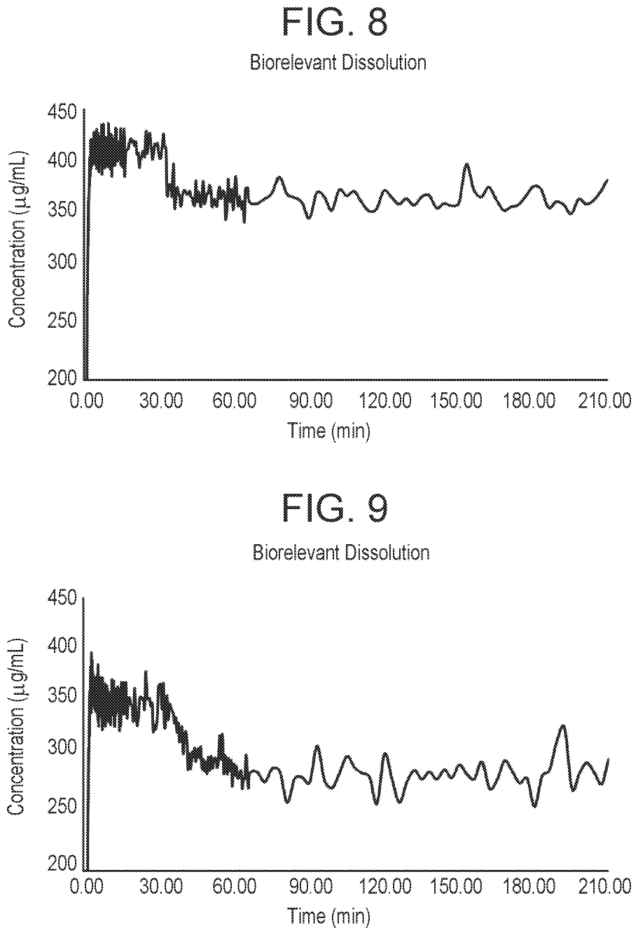

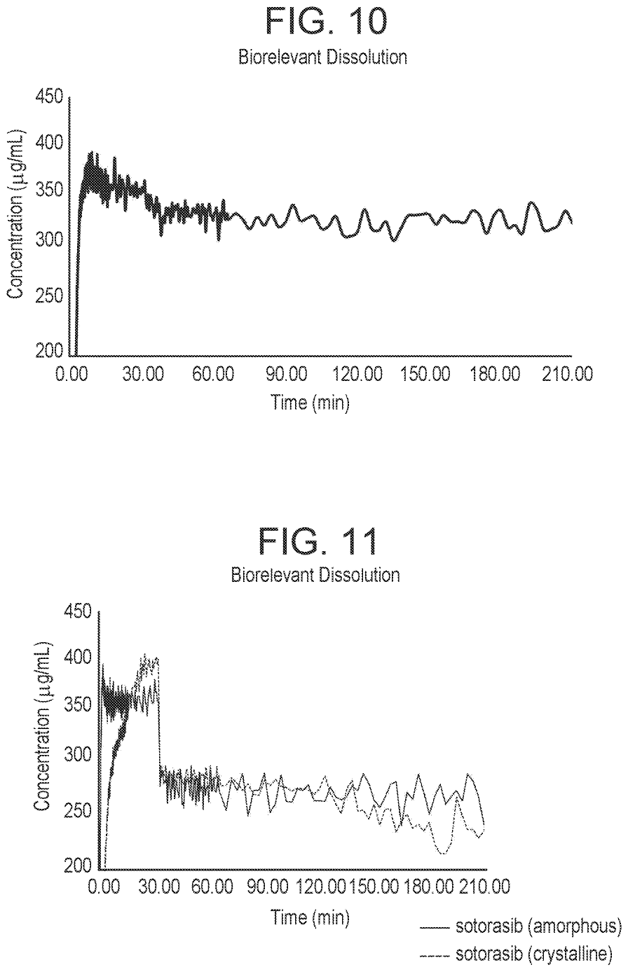

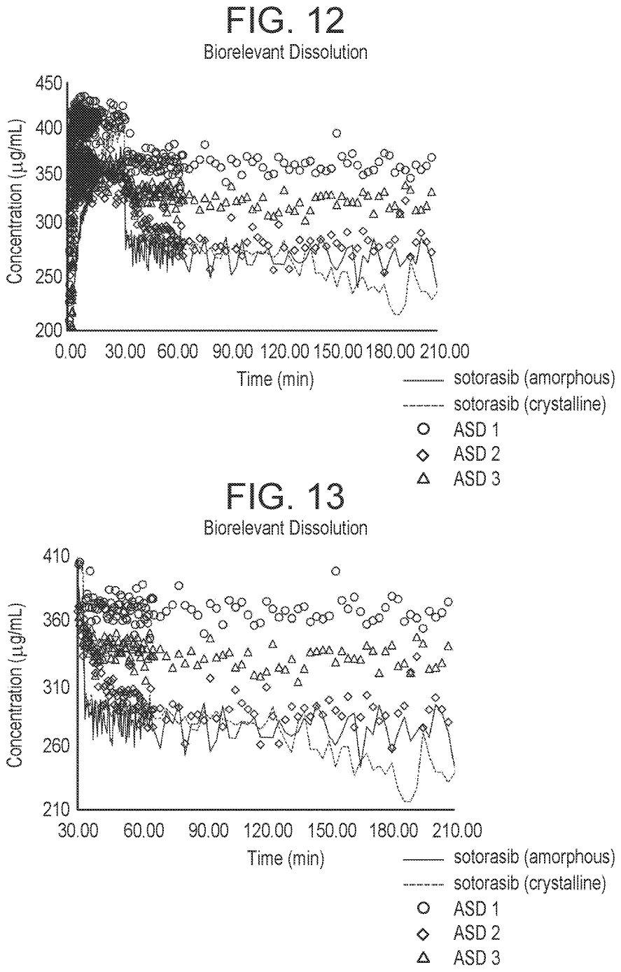

Biorelevant Dissolution: The biorelevant dissolution of ASDs 1-3 was evaluated in intestinal fluid and gastric fluid as shown in FIGS. 6-10 using amorphous sotorasib and crystalline sotorasib as references (FIGS. 6 and 7, respectively). A comparison of the biorelevant dissolution between amorphous and crystalline sotorasib is shown in FIG. 11. The biorelevant dissolution data were obtained using a Pion microdissolution apparatus in Fasted State Simulated Gastric Fluid (FaSSGF) for 30 minutes and then transitioned into Fasted State Simulated Intestinal Fluid (FaSSIF) for an additional 3 hours while monitoring at a fixed wavelength (355 nm). The biorelevant dissolution of ASDs 1-3 is shown in FIGS. 8-10. A comparison of all the biorelevant dissolution is shown in FIG. 12 (time 0-210 min) and FIG. 13 (time 30-210 min). The ASD's 1 (FIG. 8) and 3 (FIG. 10) maintained higher sotorasib solubility after transitioning into the intestinal fluid (FIGS. 12 and 13).

### Example 2—Stability Testing of ASD 1, ASD 2 and ASD 3

Stability testing of the three ASDs prepared in Example 1 was conducted over 2 months (T=0, 1 month and 2 months) using XRPD, TGA, DSC, PSD, and dissolution tests (FaSSIF and biorelevant media).

### Methods

Stability Testing: ASD samples were stored in low density polyethylene (LPDE) bags placed in a heat-sealed aluminum foil pouch along with desiccant. Foil pouches were then placed into a stability chamber at 25 °C/60% RH and taken out for stability testing (XRPD, TGA, DSC PSD, dissolution and biorelevant dissolution) at specified timepoints.

X-Ray Powder Diffraction (XRPD) Analysis: XRPD data was obtained using a Panalytical PW3040-PRO diffractometer. Samples were scanned at room temperature (25 °C) in continuous mode from 3-45 degrees (2θ) with step size of 0.03 degrees at 45 kV and 40 mA with CuKα radiation (1.54 A). The incident beam path was equipped with a primary soller slit 0.04 rad and a divergence slit 0.38 mm, in a fixed slit mode. Samples were prepared on a low background sample holder and placed on a spinning stage with a rotation time of 4 sec.

Thermal Gravimetric analysis (TGA): TGA was performed to evaluate presence for residual solvent and any thermal transitions which may result in weight loss. TGA was performed on a TA Instruments Discovery 550 Series analyzer at 5 °C/min from ambient temperature to 200 °C in a platinum pan under dry nitrogen at 25 ml/min. Sample size was ~2-8 mg.

Differential Scanning Calorimetry (DSC) Analysis: DSC data is collected using a TA Instruments Q2500 DSC. Approximately, 2-6 mg of the sample was placed in a Tzero aluminum pan and sealed with Tzero lid. Method Log consist of equilibrating to −20 °C, holding Isothermal for 1.00 min, Ramping 10.00 °C/min to 160.0 °C, holding isothermal for 1.00 min, bringing down to Ramp of 10.00 °C/min to −20.00 °C, holding isothermal for 1.00 min, ramping 10.00 °C/min to 200 °C, marking end of cycle. Conditions were done under a nitrogen purge of about 50 mL/min.

Particle Size Distribution (PSD): Analysis of each of the samples was obtained using a Sympatec Helos laser diffraction analyzer. The results are shown below (D10, D50, and D90 values).

Dissolution (FaSSIF): The dissolution properties of ASDs 1-3 and the control spray-dried sotorasib were assessed as described herein. 5 mg of amorphous sotorasib (control), 5 mg of crystalline sotorasib (control), and 20 mg of ASDs1-3 as listed in Table 1 were added to separate 20 mL vials of FaSSIF (pH 6.50) and samples were collected at 5, 15, 20, 30, 60, 120, 180 min timepoints. All crystalline samples as well as the first two timepoints of amorphous samples were not diluted due to their low solubility, the remaining 3 ASDs were diluted by 5-fold in 50:50 water:acetonitrile and run on HPLC using a C18 column as the stationary phase under the conditions shown in Table 2. The mobile phases were A:Water with 0.1% trifluoroacetic acid and B:Acetonitile with 0.1% trifluoroacetic acid. The method was a reverse gradient of 5% B 95% A to 98% B 2% A over 15 minutes using a wavelength 254 nm for detection and quantification.

Dissolution (biorelevant): The dissolution properties of ASDs 1-3 and the control spray-dried sotorasib were assessed as described herein. 10 mg of amorphous sotorasib (control), 10 mg of crystalline sotorasib (control), and 40 mg of ASDs1-3 as listed in Table 1 were added to separate 20 mL vials of FASSIF (pH 6.50) and samples were collected at 5, 10, 20, 30, 35, 40, 50, 60, 90, 120, 150, 180, and 210 min timepoints. Crystalline samples consisting of timepoint 5 min through 90 min were diluted 10-fold, timepoints of 120 min to 210 min were diluted 5-fold, Amorphous samples as well as the 3 ASDs were diluted by 10-fold in 50:50 water:acetonitrile and run on HPLC using a C18 column as the stationary phase under the conditions shown in Table 2. The mobile phases were A:Water with 0.1% trifluoroacetic acid and B:Acetonitrile with 0.1% trifluoroacetic acid. The method was a reverse gradient of 5% B 95% A to 98% B 2% A over 15 minutes using a wavelength 254 nm for detection and quantification.

### Results

The following results were obtained for control (amorphous sotorasib) and ASD 1-3:

TABLE 3
XRPD

| Material Tested | Time Point | Result |
|---|---|---|
| Control (amorphous | T = 0 | amorphous |
| sotorasib) | T = 1 month | amorphous |
| | T = 2 months | amorphous |
| ASD 1 (E PO) | T = 0 | amorphous |
| | T = 1 month | amorphous |
| | T = 2 months | amorphous |
| ASD 2 (PVP-VA) | T = 0 | amorphous |
| | T = 1 month | amorphous |
| | T = 2 months | amorphous |
| ASD 3 (HPMCAS-M) | T = 0 | amorphous |
| | T = 1 month | amorphous |
| | T = 2 months | amorphous |

Table 3 shows that ASD 1-3 remains amorphous over a period of at least two months.

TABLE 4
TGA

| Material Tested | Time Point | Result (weight change) |
|---|---|---|
| Control (amorphous | T = 0 | <1% |
| sotorasib) | T = 1 month | <1% |
| | T = 2 months | <1% |
| ASD 1 (E PO) | T = 0 | <1% |
| | T = 1 month | <1% |
| | T = 2 months | <1% |
| ASD 2 (PVP-VA) | T = 0 | <1% |
| | T = 1 month | <1% |
| | T = 2 months | <1% |
| ASD 3 (HPMCAS-M) | T = 0 | <1% |
| | T = 1 month | <1% |
| | T = 2 months | <1% |

Table 4 shows that ASD 1-3 show no relevant weight change over a period of at least two months.

TABLE 5
DSC

| Material Tested | Time Point | Result - Tg (°C) |
|---|---|---|
| Control (amorphous | T = 0 | n1: 147.7 |
| sotorasib) | | n2: 146.9 |
| | T = 1 month | n1: 148.4 |
| | | n2: 148.3 |
| | T = 2 months | n1: 149.1 |
| | | n2: 148.6 |
| ASD 1 (E PO) | T = 0 | n1: 55.0 |
| | | n2: 55.7 |
| | T = 1 month | n1: 54.7 |
| | | n2: 53.7 |
| | T = 2 months | n1: 53.8 |
| | | n2: 53.7 |
| ASD 2 (PVP-VA) | T = 0 | n1: 120.8 |
| | | n2: 120.7 |
| | T = 1 month | n1: 120.8 |
| | | n2: 120.8 |
| | T = 2 months | n1: 119.3 |
| | | n2: 119.3 |
| ASD 3 (HPMCAS-M) | T = 0 | n1: 119.9 |
| | | n2: 120.4 |
| | T = 1 month | n1: 120.0 |
| | | n2: 119.2 |
| | T = 2 months | n1: 118.9 |
| | | n2: 118.1 |

Table 5 shows that ASD 1-3 show no relevant change in Tg over a period of at least two months.

TABLE 6
PSD

| Material Tested | Time Point | Result - (D₁₀, D₅₀, D₉₀) μm |
|---|---|---|
| Control (amorphous | T = 0 | (0.8, 3.6, 13.4) |
| sotorasib) | T = 1 month | (0.8, 4.2, 14.4) |
| | T = 2 months | (0.8, 3.8, 13.5) |
| ASD 1 (E PO) | T = 0 | (2.3, 9.4, 20.4) |
| | T = 1 month | (2.4, 10.6, 23.6) |
| | T = 2 months | (2.7, 10.5, 22.0) |
| ASD 2 (PVP-VA) | T = 0 | (2.0, 11.5, 26.1) |
| | T = 1 month | (2.1, 10.5, 24.6) |
| | T = 2 months | (2.1, 10.1, 24.2) |
| ASD 3 (HPMCAS-M) | T = 0 | (3.8, 12.8, 30.9) |
| | T = 1 month | (6.9, 26.3, 48.7) |
| | T = 2 months | (5.3, 20.9, 45.0) |

Table 6 shows that ASD 1-3 show no relevant change in particle size distribution over a period of at least two months.

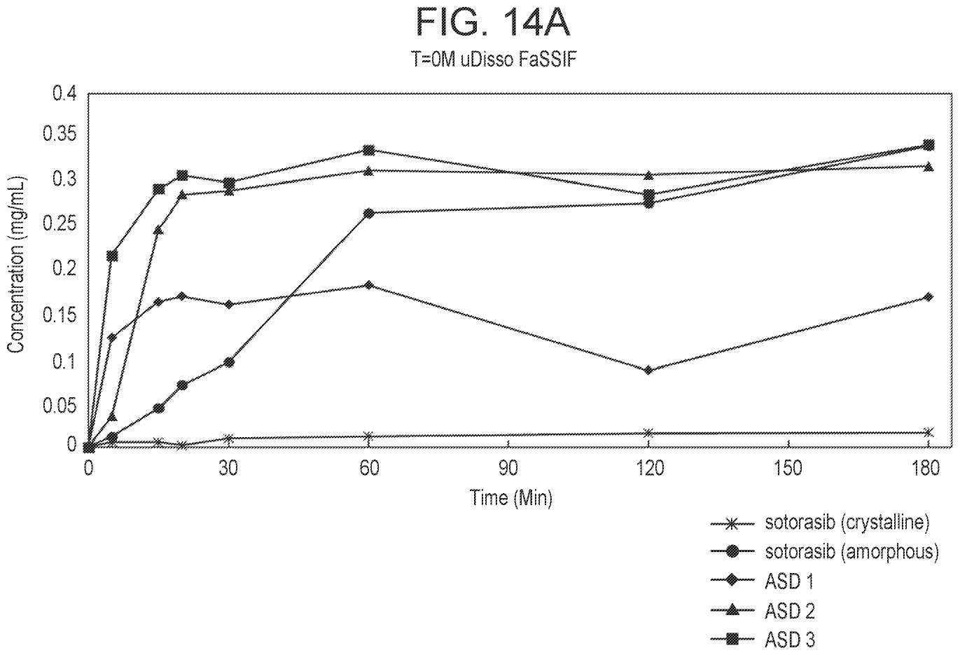

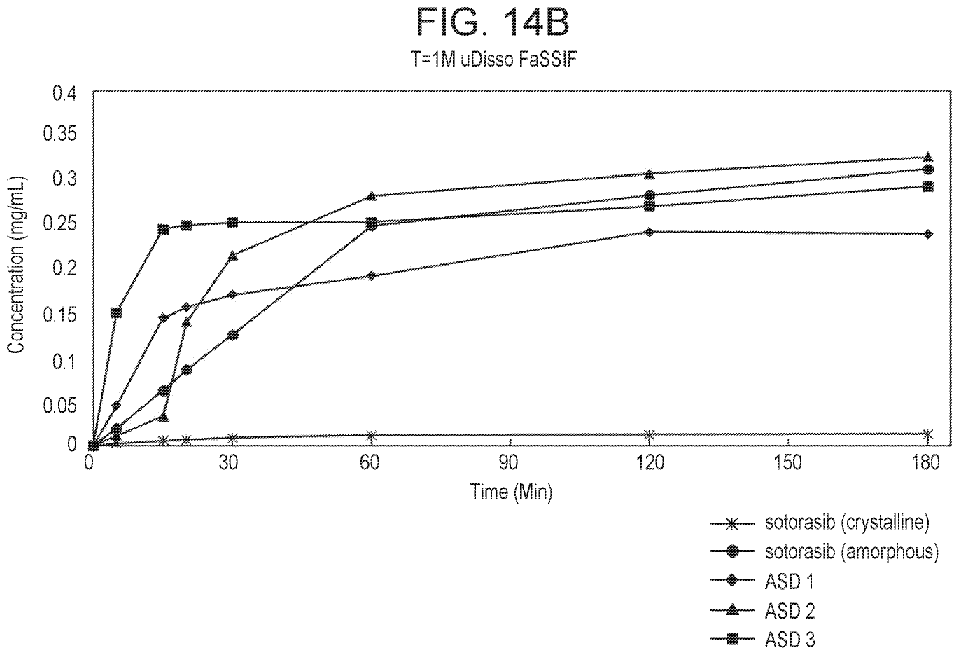

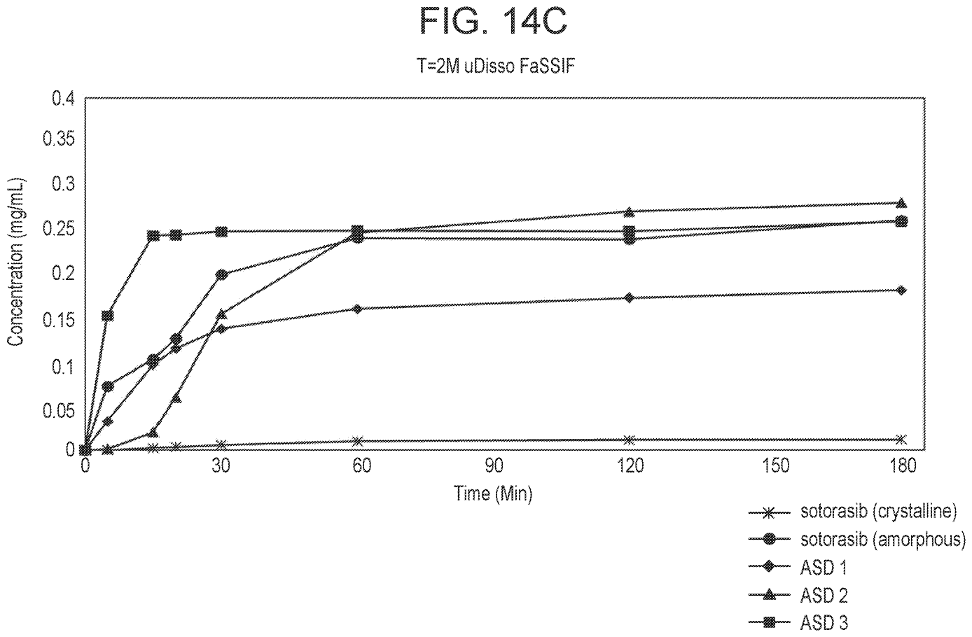

The dissolution of the ASDs 1-3 were compared against amorphous and crystalline sotorasib (AMG 510) (Form I, see WO2020/236947) at T=0, 1 month, and 2 months. The results of the dissolution study are shown in FIG. 14 and demonstrate that each of the ASDs prepared demonstrated enhanced dissolution and solubility compared to the crystalline sotorasib over the testing period, wherein the ASD comprising sotorasib and HPMCAS-M exhibited the highest dissolution.

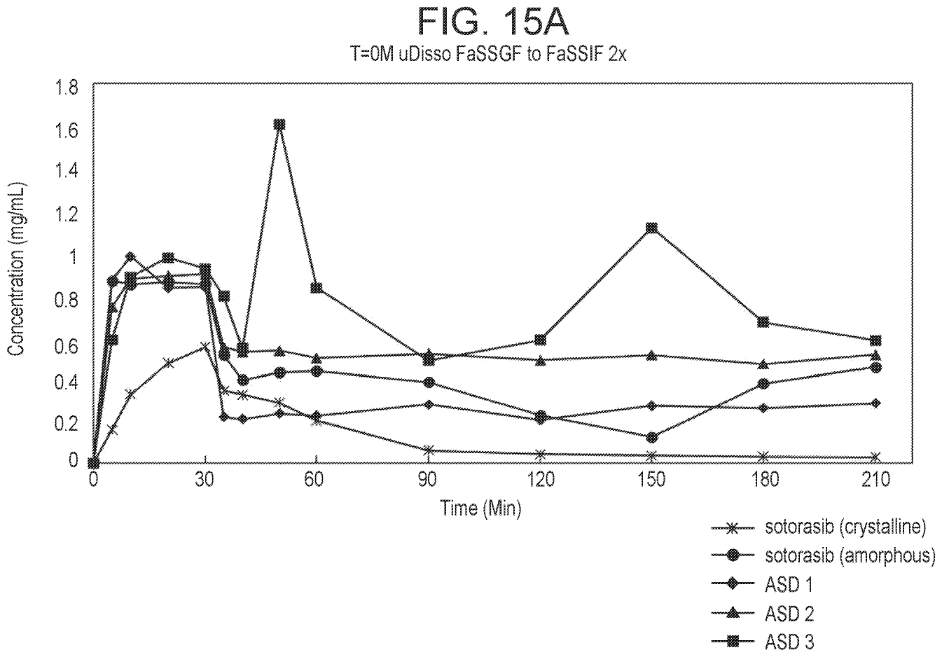

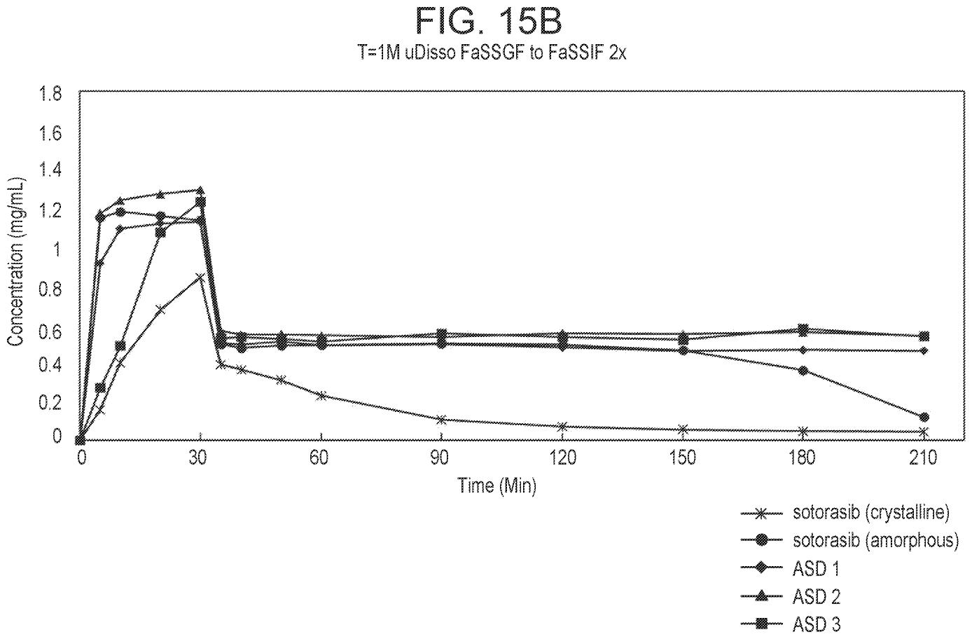

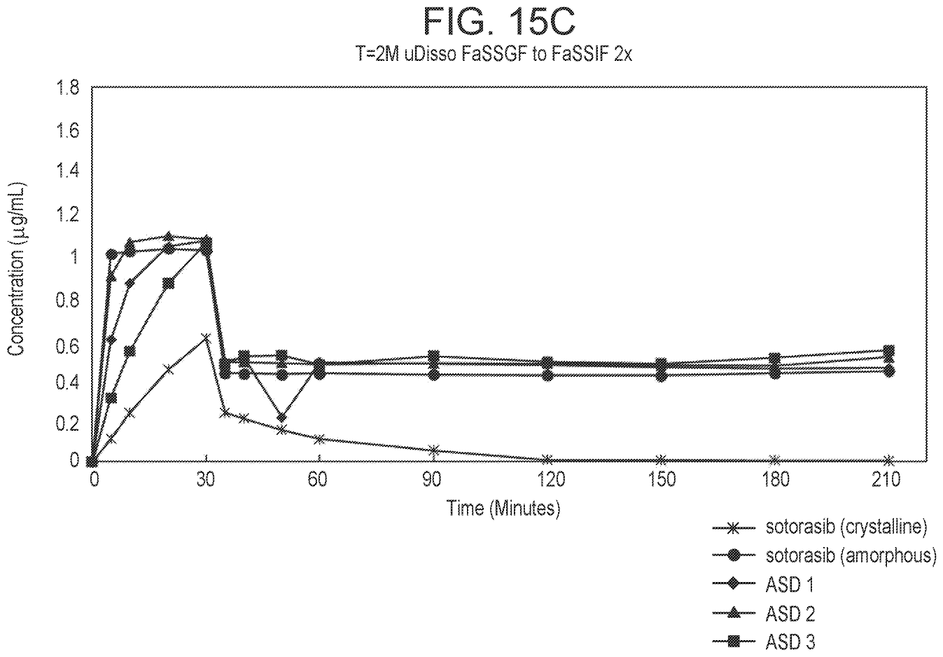

The biorelevant dissolution of ASDs 1-3 was compared against amorphous and crystalline sotorasib (Form I, see WO2020/236947) in intestinal fluid and gastric fluid as shown in FIG. 15. The results of the dissolution study demonstrate that each of the ASDs prepared maintained higher sotorasib solubility after transitioning into the intestinal fluid over the testing period.

ASD 3 (HPMCAS-M based) was used to assess compactibility and tabletability as shown in Example 3.

### Example 3—Compactibility and Tabletability

To guide the preparation of pharmaceutical compositions, such as tablets, the compactability and tabletability profile of ASD 3 was assessed.

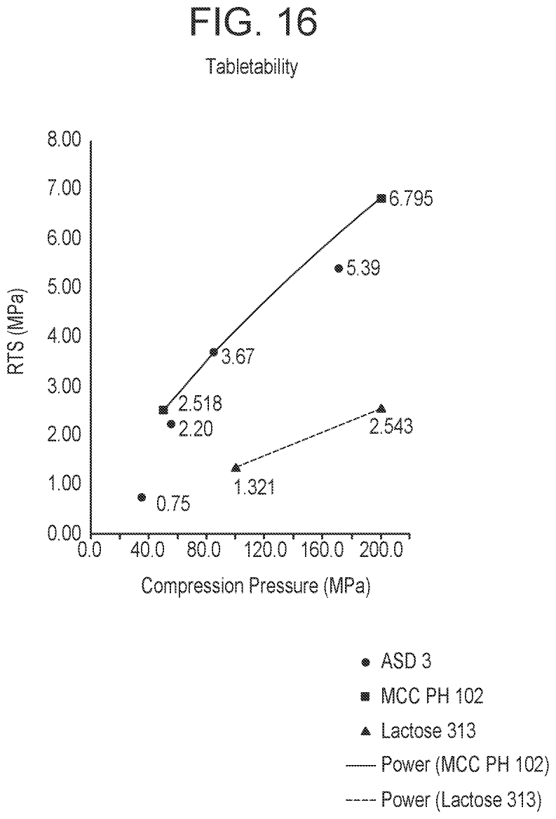

The tabletability profile explains the relationship between pressure and tablet strength. Compaction studies were performed in a Huxley Bertram HB100 compaction simulator. The simulator was equipped with 6.35 mm, B-type top and bottom punches with round, flat faces. For this assessment, ASD 3 was first compressed at 50 MPa compression pressure using a 6.35 mm die. The amount of compressed material for each data point was approximately 86-95 mg. The punch gap was adjusted manually by adjusting the lower punch to accommodate powder into the die as needed. The solid fraction, compression pressure and relative tensile strength were calculated according to common practice. The data was compared with lactose and micro-crystalline cellulose (MCC) PH 102 generated as described in Example 5 of International Patent Application Publication No. WO2022/235904. The resulting tabletability profile is presented in FIG. 16. The data shows that the material properties of ASD 3 are more similar to MCC PH 102 than lactose.

Compactibility gives additional information to describe the overall tableting behavior, keeping into account other parameters influencing the process, such as porosity (described here as solid fraction). The ability of a powder bed to cohere into or to form a compact gives an indication of powder compactibility. This behavior can be described, e.g., by a plot of tablet tensile strength as a function of tablet solid fraction. Solid fraction was calculated according to common practice (i.e., ratio of tablet density to true powder density).

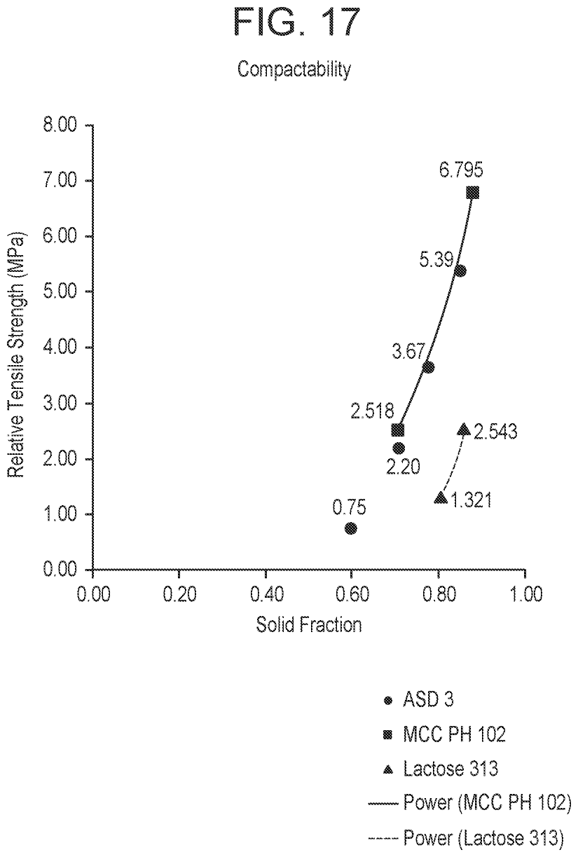

The neat ASD 3 compacts prepared at ~55 MPa and 170 MPa have a radial tensile strength of 2.20 and 5.39 respectively. These values are similar to those for MCC PH 102 (@50 MPa—RTS is 2.52 and @200 MPa—RTS is 6.80). See FIG. 17.

In conclusion, the data shows that the similarity of the material properties of ASD 3 to MCC Similarity of the AMG 510:HPMCAS-M SDD with MCC allows for reduction of MCC in favor of increasing drug load within tablet. ASD 3 was then used to prepare pharmaceutical compositions as shown in Example 4 below.

### Example 4—Pharmaceutical Compositions of ASD 3

This example describes the preparation of three pharmaceutical compositions using ASD 3. Specifically, three formulated blends comprising 4% ASD 3, 71% ASD and 94% ASD were prepared. The blend consisting of 71% ASD was subsequently tableted.

TABLE 7
Blend comprising 4% ASD 3

| Ingredient | % w/w | mg |
|---|---|---|
| ASD 3 (25% sotorasib) | 4.00 | 200.00 |
| MCC PH102 (diluent) | 69.00 | 3450.00 |
| Lactose 313 (diluent) | 23.00 | 1150.00 |
| Croscarmelose sodium (disintegrant) | 3.00 | 150.00 |
| Magnesium stearate (lubricant) | 1.00 | 50.00 |
| Total | 100.00 | 5000.00 |

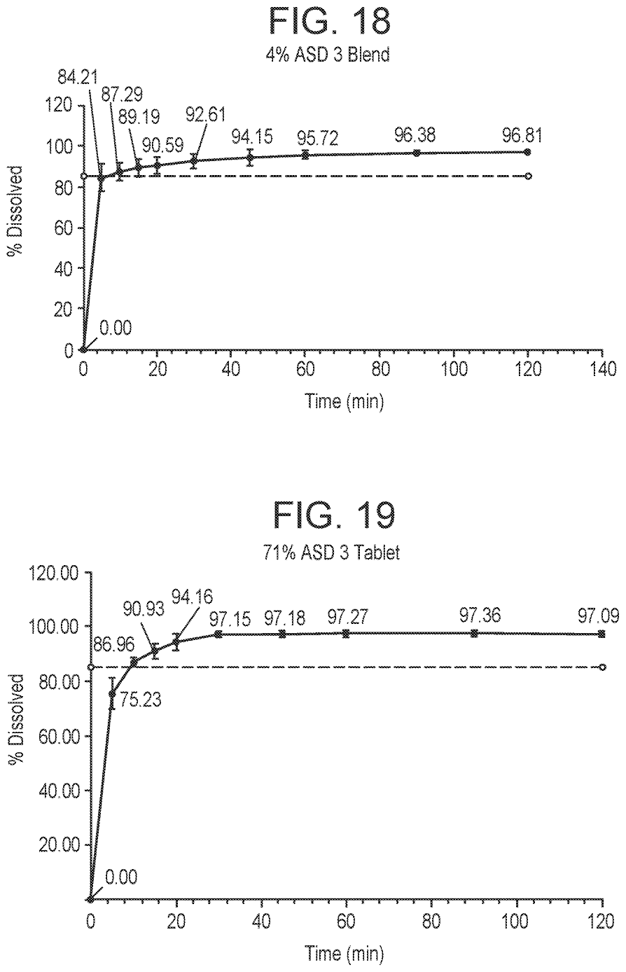

A 5 g blend was prepared using the ingredients as shown in Table 7. MCC and croscarmellose were weighed first and transferred into a plastic 20 ml scintillation vial. ASD 3 was then weighed and added to the same scintillation vial. Finally, lactose was weighed and added. The vial was mounted on an acoustic mixer (LabRAM, Resodyn Acoustic Mixers, MT, USA) and the ingredients were mixed at 75% intensity for 2 minutes. Subsequently, the blend was mixed manually with a spatula to ensure contents did not stick to vial walls. An anti-static gun was also used. Magnesium stearate was then weighed and added to the blend. The final blend was mixed further at 75% intensity for another 2 minutes. This prepared blend was used for the dissolution experiment described below. The results are shown in FIG. 18.

TABLE 8
Tablet comprising 71% ASD 3

| Ingredient | % w/w | mg/tablet |
|---|---|---|
| ASD 3 (25% sotorasib) | 70.70 | 480.0 |
| Lactose 313 (diluent) | 25.20 | 171.1 |
| Croscarmellose sodium (disintegrant) | 3.50 | 23.76 |
| Magnesium stearate (lubricant) | 0.60 | 4.074 |
| Total | 100.00 | 678.93 |

A blend for 10 tablets was prepared using the ingredients as shown in Table 8. All ingredients were weighed and transferred into a plastic 20 ml scintillation vial, except magnesium stearate. The vial was mounted on an acoustic mixer (LabRAM, Resodyn Acoustic Mixers, MT, USA) and the ingredients were mixed at 75% intensity for 2 minutes. Subsequently, the blend was mixed manually with a spatula to ensure contents did not stick to vial walls. An anti-static gun was also used. Magnesium stearate was then weighed and added to the blend. The final blend was mixed further at 75% intensity for another 2 minutes. This prepared blend was tableted as described below.

The tablets were prepared by compression on a Korsch XP1 at the following settings: insertion depth=6.5-6.7 mm; fill depth=12.0 mm, tablet target weight=678.9 mg; tooling—Natoli 13 mm Flat faced TSM B; upper punch—0.5118″; lower punch—0.5118″; and die—0.5118″. These tablets were used for dissolution experiment as described below. The results are shown in FIG. 19.

TABLE 9
Blend comprising 94% ASD 3

| Ingredient | % w/w | mg |
|---|---|---|
| ASD 3 (25% sotorasib) | 94.00 | 2100.0 |
| Croscarmellose sodium (disintegrant) | 6.00 | 134.4 |
| Total | 100.00 | 2234.4 |

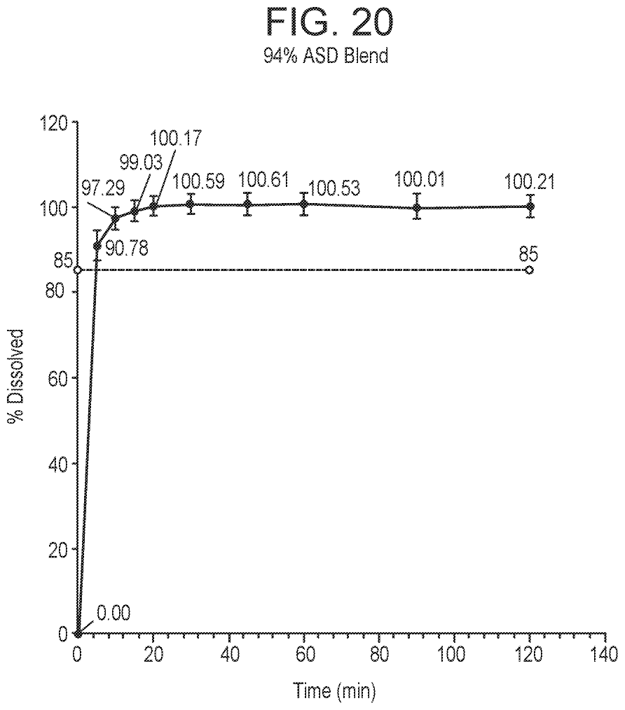

A 2.2 g blend was prepared using the ingredients as shown in Table 9. All ingredients were weighed and transferred into a plastic 20 ml scintillation vial. The vial was mounted on an acoustic mixer (LabRAM, Resodyn Acoustic Mixers, MT, USA) and the ingredients were mixed at 75% intensity for 2 minutes. Subsequently, the blend was mixed manually with a spatula to ensure contents did not stick to vial walls. An anti-static gun was also used. The final blend was mixed further at 75% intensity for another 2 minutes. This prepared blend was used for the dissolution experiment described below. The results are shown in FIG. 20.

Dissolution: The dissolution properties of a tablet comprising 71% ASD were assessed as described herein. Three tablets with a target weight of 678.925 mg were added to separate 900 mL vessels (USP Apparatus II) of 50 mM Phosphate Buffer pH 6.8 with 0.2% SDS w/w and samples were collected at 0, 5, 10, 15, 20, 30, 45, 60, 90, and 120 min timepoints (increased RPM to 250 from 90 min to 120 min). Each sample was run on HPLC using a C18 column as the stationary phase under the conditions shown in Table 10. The mobile phase was water and acetonitrile with 0.1% trifluoroacetic acid. The method was 32% acetonitrile 68% over 6 minutes using a wavelength 245 nm for detection and quantification.

TABLE 10
HPLC method for dissolution studies

| HPLC System: | Waters | |
|---|---|---|
| Data Capture System: | Empower | |
| Column: | Waters XBridge C18, 150 × 4.6 mm, 3.5 μm | |
| Column Cartridge: | XBridge BEH C18 5 × 3.9 mm, 3.5 μm | |
| Column Cartridge Holder: | Waters P/N: 186007949 | |
| Mobile Phase: | D.I. Water:ACN:TFA = 68:32:0.1, v/v/v | |
| Channel Monitoring | Pressure | |
| Sample Tray Temperature: | Ambient | |
| Column Temperature: | 30 | °C |
| Injection Volume: | 5 | μL |
| Flow rate: | 1.2 | mL/min |
| Bandwidth: | 8 | nm |
| UV Detection Wavelength: | 254 | nm |
| Run Time: | 6 | min |

| | |
|---|---|
| Needle Wash: | No |

The dissolution properties of a blend comprising 4% ASD were assessed as described hereinabove. A target weight of 500.00 mg (4% SDD) was added to three separate 150 mL vessels of 50 mM Phosphate Buffer pH 6.8 with 0.2% SDS w/w and samples were collected at 0, 5, 10, 15, 20, 30, 45, 60, 90, and 120 min timepoints (increased RPM to 250 from 90 min to 120 min). Each sample was run on HPLC using a C18 column as the stationary phase under the conditions shown in Table 10. The mobile phase was water and acetonitrile with 0.1% trifluoroacetic acid. The method was 32% acetonitrile 68% over 6 minutes using a wavelength 245 nm for detection and quantification.

The dissolution properties of a blend comprising 94% ASD were assessed as described herein. A target weight of 319.1 mg was added to three separate 900 mL vessels of 50 mM Phosphate Buffer pH 6.8 with 0.2% SDS w/w and samples were collected at 0, 5, 10, 15, 20, 30, 45, 60, 90, and 120 min timepoints (increased RPM to 250 from 90 min to 120 min). Each sample was run on HPLC using a C18 column as the stationary phase under the conditions shown in Table 10. The mobile phase was water and acetonitrile with 0.1% trifluoroacetic acid. The method was 32% acetonitrile 68% over 6 minutes using a wavelength 245 nm for detection and quantification.

1. An amorphous solid dispersion (ASD) comprising sotorasib and at least one polymer selected from the group consisting of a cationic acrylate copolymer, a vinylpyrrolidone-acetate copolymer, a cellulose polymer, and a combination thereof.

2. The ASD of claim 1, wherein sotorasib is essentially free of crystalline sotorasib.

3. The ASD of claim 1, which is devoid of crystalline sotorasib in an amount detectable by XRPD.

4. The ASD of claim 1, wherein the at least one polymer comprises a cationic acrylate copolymer.

5. The ASD of claim 4, wherein the cationic acrylate copolymer comprises a polymethacrylate polymer.

6. The ASD of claim 1, wherein the cationic acrylate copolymer comprises a copolymer based on monomers comprising aminoalkyl methacrylate and alkyl methacrylate.

7. The ASD of claim 1, wherein the cationic acrylate copolymer comprises a copolymer based on monomers comprising 2-dimethylaminoethyl methacrylate and an alkyl methacrylate.

8. The ASD of claim 1, wherein the cationic acrylate copolymer comprises a copolymer based on monomers comprising 2-dimethylaminoethyl methacrylate, butyl methacrylate, and methyl methacrylate.

9. The ASD of claim 8, wherein the cationic acrylate copolymer is based on monomers comprising 2-dimethylaminoethyl methacrylate, butyl methacrylate, and methyl methacrylate with a monomer ratio of 2:1:1.

10. The ASD of claim 1, wherein the cationic acrylate copolymer is poly((2-dimethylaminoethyl) methacrylate, butyl methacrylate, methyl methacrylate) (2:1:1).

11. The ASD of claim 1, wherein the vinylpyrrolidone-acetate copolymer comprises a copolymer based on monomers comprising N-vinyl lactam monomers and vinyl acetate monomers.

12. The ASD of claim 11, wherein the N-vinyl lactam monomers comprise N-vinyl pyrrolidone.

13. The ASD of claim 12, wherein the acetate monomers comprise vinyl acetate.

14. The ASD of claim 11, wherein the polymer comprises one or more polymers selected from povidone, copovidone, polyvinyl acetate, and a combination thereof.

15. The ASD of claim 11, wherein the ratio of N-vinyl lactam monomers to acetate monomers is 6:4.

16. The ASD of claim 1, wherein the at least one polymer comprises a random copolymer.

17. The ASD of claim 1, wherein the at least one polymer comprises one or more cellulose polymer selected from the group consisting of hydroxypropylmethyl cellulose, hydroxypropylmethyl cellulose acetate succinate, hydroxypropyl methylcellulose phthalate (HPMCP), hydroxypropyl cellulose, and a combination thereof.

18. The ASD of claim 24, wherein the cellulose polymer comprises hydroxypropylmethyl cellulose acetate succinate.

19. The ASD of claim 1, wherein the ASD has a D50 of 3 μm to 10 μm.

20.-21. (canceled)

23. The ASD of claim 19, wherein the ASD has a D90 of 10 μm to 20 μm.

24. The ASD of claim 17, wherein the ASD has a D50 of 10 μm to 30 μm.

25. (canceled)

26. The ASD of claim 24, wherein the ASD has a D10 of 3 μm to 7 μm.

27. The ASD of claim 26, wherein the ASD has a D90 of 30 μm to 50 μm.

28. The ASD of claim 1, having a weight ratio of sotorasib to the polymer of 1:50 to 10:1.

29.-30. (canceled)

31. The ASD of claim 1, wherein sotorasib is 10-80% by weight of the ASD.

32. (canceled)

33. A process for making an amorphous drug dispersion (ASD) comprising:
(a) mixing sotorasib, one or more polymers, and a solvent to form a solution; and
(b) spray-drying the solution of step (a), thereby obtaining the ASD.

34.-41. (canceled)

42. An ASD prepared by the process according to claim 33.

43.-44. (canceled)

45. A pharmaceutical composition comprising the ASD of claim 1 and a pharmaceutically acceptable excipient.

46.-68. (canceled)

69. A method of treating cancer in a patient, the method comprising administering to the patient a therapeutically effective amount of sotorasib provided as the ASD of claim 1.

70. The method of claim 69, wherein one or more cells of the cancer express a KRAS G12C mutant protein.

71. The method of claim 69, wherein the cancer is non-small cell lung cancer, colorectal cancer, pancreatic cancer, appendiceal cancer, endometrial cancer, cancer of unknown primary, ampullary cancer, gastric cancer, small bowel cancer, sinonasal cancer, bile duct cancer, or melanoma.

72.-73. (canceled)
